(19) United States
(12) Patent Application Publication
(10) Pub. No.: US 2026/0131045 A1
(43) Pub. Date: May 14, 2026

(54) Biocompatible Material

(71) Applicant: Trimph IP Pty Ltd, Sydney (AU)

(72) Inventors: Ali FATHI, Northmead (AU); Terence ABRAMS, Eveleigh (AU); Dax CALDER, Eveleigh (AU)

(21) Appl. No.: 19/445,229

(22) Filed: Jan. 9, 2026

Related U.S. Application Data
(63) Continuation of application No. 17/787,144, now abandoned.

(30) Foreign Application Priority Data
Dec. 19, 2019 (AU) 2019904817
Sep. 25, 2020 (AU) 2020903462

(51) Int. Cl. A61L 24/06; A61L 24/10; A61L 27/16; A61L 27/22; A61L 27/26; A61L 27/52

(52) U.S. Cl. CPC A61L 24/06; A61L 24/10; A61L 27/16; A61L 27/22; A61L 27/26; A61L 27/52

(57) ABSTRACT

The present invention provides a composition comprising a polymer and a natural or synthetic peptide or protein (NSPP). The composition forms a hydrogel with water. The composition is useful as a filler for cosmetic and therapeutic applications. Embodiments of the invention provide methods of treating certain conditions using the composition or hydrogel, and surgical kits for the simultaneous or sequential administration of the respective components of the composition, enabling the formation of the hydrogel in situ.

### CROSS-REFERENCE TO RELATED APPLICATIONS

This application is a continuation of U.S. patent application Ser. No. 17/787,144, filed Jun. 17, 2022, which is a National Stage Entry under 35 U.S.C. § 371 of International Patent Application No. PCT/AU2020/051332, filed Dec. 7, 2020. The foregoing applications are incorporated by reference herein in their entireties.

### FIELD OF THE INVENTION

The present invention relates to a biocompatible material. The biocompatible material is useful in tissue regeneration and repair.

The present invention relates to a filler that supports the natural healing of damaged tissue without inducing any specific tissue-formation. It is aimed to use the invention to partially or completely fill or cover a tissue cavity or defect to provide required filling space with minimal foreign body reaction.

In one embodiment, the present invention relates to a tissue conductive medical filler. In one embodiment, the polymer of the present invention can be formulated as a hydrogel. In another embodiment, the hydrogel is thermoresponsive. In yet another embodiment, the compositions disclosed herein have been developed for delivery in a flowable form which can be either injected, pored or sprayed. In an embodiment, the compositions form hydrogels after administration into or onto or adjacent the body.

The present invention is useful in tissue engineering applications. This includes both cosmetic and therapeutic applications. The present invention is useful in dermal applications as well as dental and orthopaedic applications for the treatment of chronic, acute or surgically-created defects. However, it will be appreciated that the invention is not limited to these particular fields of use.

### BACKGROUND OF THE INVENTION

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field, or that this prior art could reasonably be expected to be ascertained, understood and regarded as relevant by a person skilled in the art.

All of the patents and publications referred to herein are incorporated by reference in their entirety.

WO 2013/091001 (PCT/AU2012/001566) relates to polymers, especially polymers useful as hydrogels, and to use of hydrogels for repair or restoration of tissue. In particular, the polymers and hydrogels of WO 2013/091001 can be used for the repair or restoration of cartilage, especially articular cartilage. The polymers comprise at least a monomer for binding water, a monomer for imparting mechanical properties and a monomer for binding to an extracellular protein. The hydrogels comprise a polymer comprising at least a monomer for binding water and a monomer for binding to an extracellular protein. Crosslinking polymers by binding of said extra-cellular matrix protein forms hydrogels.

WO 2017/035587 (PCT/AU2016/050817) discloses biocompatible materials useful for tissue regeneration and repair, wherein the bioactive polymer may be in the form of a hydrogel, for example a thermoresponsive hydrogel. The bioactive polymer and resulting hydrogel of WO 2017/035587 may be used for the regeneration of bone tissue. Accordingly, WO 2017/035587 discloses methods of treating a bone defect in a mammal, the methods comprising administering a therapeutically effective amount of a hydrogel formed by the bioactive polymer to the mammal to treat the bone defect.

WO 2017/015703 (PCT/AU2016/050653) discloses a polymer comprising at least one antiseptic/analgesic/anti-inflammatory monomeric unit in conjunction with at least three further monomeric units, said three further monomeric units eliciting properties selected from the group consisting of: temperature activation, water solubility, mechanical strength, protein/polysaccharide bonding capacity, and combinations thereof. In particular, WO 2017/015703 discloses a polymer, wherein: the water-soluble monomeric unit is a hydrophilic ethylene glycol (OEGMA) moiety; the mechanical strength-conferring monomeric unit is polylactide-co-2-hydroxy-ethylmethyl acrylate (PLA/HEMA); the protein-reactive monomeric unit is an N-acryloxysuccinimide (NAS) moiety; and the thermosetting monomeric unit is an N-isopropyl acrylamide (NIPAAm) moiety. The antiseptic/analgesic/anti-inflammatory monomeric unit comprises a methacrylic ester derivative of salicylic acid (5-HMA or 4-HMA, or a combination thereof).

All three applications referenced above are assigned to the present Applicant. It is against this background that the present invention has been developed.

In one form, the present invention is embodied as a flowable filler, wherein upon administration into the body (e.g., by injection) or onto the body surface (i.e., at 30-37° C.) the filler forms an adhesive hydrogel. The hydrogel is well-tolerated in the body with minimal inflammatory response. The hydrogel is host tissue-conductive but not inductive as it only displays regenerative properties in the presence of an active bleeding or other fluids containing regenerative biological components. The filler can be injected through a fine gauge needle (e.g., 21 G). The hydrogel adheres to the injection site. The filler can be formulated within aerosols for administration via spray. The hydrogel can be administered in a manner that creates a 3D structure; layer-by-layer inside the body or topically through a minimally invasive manner. The principal innovative aspects of the inventive filler include minimal foreign body reaction, host-tissue conductivity, mixing with blood, injectability, adhesion characteristics, layer-by-layer filling and an optimal degradation profile.

The present invention is useful for soft tissue applications, such as dermal applications, and hard tissue applications, such as dental and orthopaedic. For example, the present invention is useful for cosmetic applications such as wrinkle reduction. The present invention is useful for promoting scar healing. This includes scars from burns as well as post-surgical scars. The invention can also be used for the management of chronic wounds such as diabetic ulcers.

The present invention is also useful for dental applications. Tooth extraction is an inherently traumatic procedure that damages the soft tissue, underlying bone and ultimately leads to significant loss of jaw or alveolar bone. Clinically, the loss of alveolar bone results in aesthetic and functional complications in relation to future prosthetic replacement of the missing tooth. If the missing tooth is to be replaced with implant-supported restoration, complex bone grafting procedures are invariably required. In an effort to reduce or potentially eliminate complex bone grafting procedures, socket or ridge preservation techniques have been suggested.

A number of techniques have been described in the literature, most of which involve the placement of grafting filler materials within the tooth extraction socket immediately following tooth removal. There are a number of commercially available products that have been used to fill extraction. Indeed, none of the currently available products can restore bone volume to pre-extraction levels or result in improved healing outcomes.

Clinical studies with demineralised bone allografts, synthetic bioactive glass and deproteinised bovine bone minerals (xenografts) have shown that even 6-9 months post administration, the grafting particles were surrounded by connective tissues or woven-like bony environment. These findings demonstrate that the healing has been physically hindered by the grafting particles. The resulting operation site even after the protracted waiting period is an unpredictable surgical environment for implant placement. Given the scope of the problem associated with bone loss following tooth extraction, there is a significant unmet need for a scaffold to improve bone healing outcomes for patients undergoing tooth extraction.

There is a general need for compositions for repair of tissue that are injectable at room temperature and that form a hydrogel at body temperature.

The composition of the present invention is intended to be used as a filler that supports the natural healing of damaged tissue without inducing any specific tissue-formation. It is aimed to use the invention to fill a tissue cavity, either partially or completely, to provide required filling space with minimal foreign body reaction. The invention can adhere to the cavity, mixes with blood to facilitate host soft and hard tissue repair. To induce any specific tissue growth, e.g., soft or hard, the invention can be used in combination with other active ingredients.

It is an object of the present invention to overcome or ameliorate at least one of the disadvantages of the prior art, or to provide a useful alternative.

It is an object of an especially preferred form of the present invention to use PNPHO-co-TB4, at specific concentrations and in specific formulations as a filler material. The filler material does not have any tissue inductive properties. The filler can be used in combination with other materials, e.g., inactive to provide 3D structure, or active to induce a formation of a specific tissue. The invention can be injected, poured or sprayed. The concentration of polymer and TB4 can be adjusted to form different form of the invention.

The invention is intended to be used for soft and hard tissue. In respect of soft tissue, the invention is added to a bioactive ingredient (like cells or fat grafts) for skin type applications; the product can be injected or sprayed. For hard tissue or hard/soft tissue applications, the invention can be applied with a 3D filler (e.g., inactive bone particles) or with active compound (e.g., growth factors) to promote both soft and hard tissue growth. The commercial form envisaged is either an injectable or a putty.

Although the present invention will be described hereinafter with reference to its preferred embodiment, it will be appreciated by those skilled in the art that the spirit and scope of the invention may be embodied in many other forms.

### SUMMARY OF THE INVENTION

In the present invention, Applicant has optimised its proprietary smart polymer, PNPHO, to bond with Thymosin beta-4 to form a cell-friendly medical filler material.

According to a first aspect of the present invention there if provided a composition comprising a polymer and a natural or synthetic peptide or protein (NSPP), wherein the polymer comprises:
a first monomer for binding water;
a second monomer for imparting mechanical properties to said hydrogel;
a third monomer for binding to a natural or synthetic peptide or protein (NSPP); and
a fourth monomer for imparting phase-transition behaviour;
and wherein the natural or synthetic peptide or protein (NSPP) is Thymosin beta-4 or a functional homolog thereof.

In an embodiment, the first monomer is selected from: polyethers, polyvinyl alcohol (PVA); poly(vinyl pyrrolidone) (PVP); poly(amino acids) and dextran.

In an embodiment, the polyethers are selected from: polyethylene glycol (PEG), oligo(ethylene glycol) (OEG) or macromonomers thereof, polyethylene oxide (PEO), polyethylene oxide-co-propylene oxide (PPO), co-polyethylene oxide block or random copolymers thereof.

In an embodiment, the first monomer is oligo (ethylene) glycol monomethyl ether methacrylate (OEGMA).

In an embodiment, the second monomer is a methacrylate, or a random co-polymer comprising a methacrylate.

In an embodiment, the second monomer is selected from: hydroxyethyl methacrylate (HEMA), a hydroxyethyl methacrylate poly(lactic acid) copolymer (HEMA-PLA), poly(lactic acid), poly(caprolactone), poly(glycolide), poly(glycolide-co-lactide) or poly(glycolide-co-caprolactone).

In an embodiment, the second monomer is hydroxyethyl methacrylate poly(lactic acid) (HEMA-PLA).

In an embodiment, the third monomer has electrophilic functional groups for binding to said NSPP.

In an embodiment, the third monomer is selected from: N-hydroxysulfosuccinimide (SNHS), N-hydroxyethoxylated succinimide (ENHS), and N-acryloxysuccinimide (NAS).

In an embodiment, the third monomer is N-acryloxysuccinimide (NAS). In an embodiment, the fourth monomer is selected from: poly(ethylene oxide)/poly(propylene oxide) and poly(N-isopropylacrylamide) (PNIPAAm) homopolymers and copolymers.

In an embodiment, the fourth monomer is (N-isopropylacrylamide). In an embodiment, the polymer comprises the first monomer in an amount of from about 3 to about 8 mol %.

In an embodiment, the polymer comprises the second monomer in an amount of from about 5 to about 9 mol %.

In an embodiment, the polymer comprises the third monomer in an amount of at least about 7 mol %, such as 8, 9, 10, 11, 12, 13, 14, 15, 16, 17, 18 or 19 mol %.

In an embodiment, the polymer comprises: the first monomer in an amount of from about 3 to about 8 mol %, the second monomer in an amount of from about 5 to about 9 mol %, the third monomer in an amount of at least about 7 mol %, and the fourth monomer in an amount which makes up the remainder to 100% of the polymer composition.

In an embodiment, the polymer comprises the fourth monomer in an amount from about 60 to about 85 mol %, preferably, about 64, 65, 66, 67, 68, 69, 70, 71, 72, 73, 74, 75, 76, 77, 78, 79, 80, 81, 82, 83, 84 or 85 mol %.

In an embodiment, the first monomer is OEGMA, the second monomer is HEMA-PLA, the third monomer is NAS, and the fourth monomer is NIPAAm, and wherein the polymer comprises: OEGMA in an amount of from about 3 to about 8 mol %, HEMA-PLA in an amount of from about 5 to about 9 mol %, NAS in an amount of greater than about 7 mol % and NIPAAm in an amount of up to about 85 mol %.

In an embodiment, the polymer comprises: OEGMA in an amount of about 5 mol %, HEMA-PLA in an amount of about 7 mol %, NAS in an amount of greater than about 7 mol % and NIPAAm in an amount about 81 mol %.

In an embodiment, the natural or synthetic peptide or protein (NSPP) is Thymosin beta-4.

In an embodiment, the composition comprises essentially equimolar amounts of the polymer and Thymosin beta-4.

In an embodiment to partially or completely fill a cavity, the concentration of the polymer is from about 100 mg/mL to about 300 mg/mL of the composition.

According to a second aspect of the present invention there is provided a hydrogel comprising the composition according to the first aspect of the present invention and water, wherein the binding of the NSPP to the third monomer crosslinks the polymer, thereby forming a hydrogel, with the water contained therein.

According to a third aspect of the present invention there is provided a method of making a hydrogel, the method comprising adding water to the composition of the first aspect of the invention.

According to a fourth aspect of the present invention there is provided a method of making a hydrogel, the method comprising mixing an aqueous solution of the composition of the first aspect of the present invention with an aqueous solution of the natural or synthetic peptide or protein (NSPP).

In an embodiment, the hydrogel is formed at body temperature. In an embodiment, the hydrogel is formed following administration of the composition and the NSPP to a mammal by injection or by administering an aerosol.

According to a fifth aspect of the present invention there is provided use of a composition according to the first aspect of the present invention in the manufacture of a hydrogel for repair and/or restoration of both hard and soft tissue. Hard tissue (also termed calcified tissue) is tissue which is mineralised and has a firm intercellular matrix; the hard tissues of humans are bone, tooth enamel, dentin, and cementum. Soft tissue includes the tissues that connect, support, or surround other structures and organs of the body, not being hard tissue such as bone. Soft tissue includes tendons, ligaments, fascia, skin, fibrous tissues, fat, and synovial membranes (which are connective tissue), and muscles, nerves and blood vessels (which are not connective tissue).

According to a sixth aspect of the present invention there is provided use of a composition according to the first aspect of the present invention in the manufacture of a hydrogel for wound healing.

According to a seventh aspect of the present invention there is provided use of a composition according to the first aspect of the present invention in the manufacture of a hydrogel for temporary wrinkle reduction.

According to an eighth aspect of the present invention there is provided use of a composition according to the first aspect of the present invention in the manufacture of a hydrogel for temporarily lifting the base of a scar and promoting healing.

According to a ninth aspect of the present invention there is provided use of a composition according to the first aspect of the present invention in the manufacture of a hydrogel for supporting dermal connective tissue formation in scar tissue after a surgical intervention and promoting healing

According to a tenth aspect of the present invention there is provided use of a composition according to the first aspect of the present invention in the manufacture of a hydrogel for supporting dermal connective tissue formation in scar management of post burn injuries.

According to an eleventh aspect of the present invention there is provided use of a composition according to the first aspect of the present invention in the manufacture of a hydrogel for supporting vascular ingrowth in an acute dermal defect with bleeding and promoting healing.

According to a twelfth aspect of the present invention there is provided use of a composition according to the first aspect of the present invention in the manufacture of a hydrogel for filling a surgically generated dermal cavity.

According to a thirteenth aspect of the present invention there is provided use of a composition according to the first aspect of the present invention in the manufacture of a hydrogel for supporting a skin grafting operation.

According to a fourteenth aspect of the present invention there is provided use of a composition according to the first aspect of the present invention in the manufacture of a hydrogel for physically delivering bone graft substitutes.

According to a fifteenth aspect of the present invention there is provided use of a composition according to the first aspect of the present invention in the manufacture of a hydrogel for filling a prosthetic.

According to a sixteenth aspect of the present invention there is provided use of a composition according to the first aspect of the present invention in the manufacture of a hydrogel for use as a filler with no tissue-inductive properties.

According to a seventeenth aspect of the present invention there is provided use of a composition according to the first aspect of the present invention in the manufacture of a hydrogel for supporting and repairing periodontal tissue after tooth extraction.

According to an eighteenth aspect of the present invention there is provided use of a composition according to the first aspect of the present invention in the manufacture of a hydrogel for temporarily lifting a periodontal ligament tissue and/or supporting periodontal ligament tissue grafting.

According to a nineteenth aspect of the present invention there is provided use of a hydrogel according to the second aspect of the present invention in the manufacture of a medicament for repair and/or restoration of tissue.

According to a twentieth aspect of the present invention there is provided a method of repair and/or restoration of tissue, the method comprising administering to a mammal a composition according to the first aspect of the present invention. In an embodiment, the administration step is made by injection or by administering an aerosol, thereby to form a hydrogel at the body temperature of the mammal.

According to a twenty-first aspect of the present invention there is provided a method of wound healing, the method comprising administering to a mammal a composition according to the first aspect of the present invention. In an embodiment, the administration step is made by injection or by administering an aerosol, thereby to form a hydrogel at the body temperature of the mammal.

According to a twenty-second aspect of the present invention there is provided a method of temporary wrinkle reduction, the method comprising administering to a mammal a composition according to the first aspect of the present invention. In an embodiment, the administration step is made by injection or by administering an aerosol, thereby to form a hydrogel at the body temperature of the mammal.

According to a twenty-third aspect of the present invention there is provided a method of temporarily lifting the base of a scar and promoting healing, the method comprising administering to a mammal a composition according to the first aspect of the present invention. In an embodiment, the administration step is made by injection or by administering an aerosol, thereby to form a hydrogel at the body temperature of the mammal.

According to a twenty-fourth aspect of the present invention there is provided a method of supporting dermal connective tissue formation in scar tissue after a surgical intervention and promoting healing, the method comprising administering to a mammal a composition according to the first aspect of the present invention. In an embodiment, the administration step is made by injection or by administering an aerosol, thereby to form a hydrogel at the body temperature of the mammal.

According to a twenty-fifth aspect of the present invention there is provided a method of supporting dermal connective tissue formation in scar management of post burn injuries, the method comprising administering to a mammal a composition according to the first aspect of the present invention. In an embodiment, the administration step is made by injection or by administering an aerosol, thereby to form a hydrogel at the body temperature of the mammal.

According to a twenty-sixth aspect of the present invention there is provided a method of supporting vascular ingrowth in an acute dermal defect with bleeding and promoting healing, the method comprising administering to a mammal a composition according to the first aspect of the present invention. In an embodiment, the administration step is made by injection or by administering an aerosol, thereby to form a hydrogel at the body temperature of the mammal.

According to a twenty-seventh aspect of the present invention there is provided a method of filling a surgically generated dermal cavity, the method comprising administering to a mammal a composition according to the first aspect of the present invention. In an embodiment, the administration step is made by injection or by administering an aerosol, thereby to form a hydrogel at the body temperature of the mammal.

According to a twenty-eighth aspect of the present invention there is provided a method of supporting a skin grafting operation, the method comprising administering to a mammal a composition according to the first aspect of the present invention. In an embodiment, the administration step is made by injection or by administering an aerosol, thereby to form a hydrogel at the body temperature of the mammal.

According to a twenty-ninth aspect of the present invention there is provided a method of physically delivering bone graft substitutes, the method comprising administering to a mammal a composition according to the first aspect of the present invention. In an embodiment, the administration step is made by injection or by administering an aerosol, thereby to form a hydrogel at the body temperature of the mammal.

According to a thirtieth aspect of the present invention there is provided a method of filling a prosthetic, the method comprising administering to a mammal a composition according to the first aspect of the present invention. In an embodiment, the filler has no tissue-inductive properties. In an embodiment, the administration step is made by injection or by administering an aerosol, thereby to form a hydrogel at the body temperature of the mammal.

According to a thirty-first aspect of the present invention there is provided a method of supporting and repairing periodontal tissue after tooth extraction, the method comprising administering to a mammal a composition according to the first aspect of the present invention. In an embodiment, the administration step is made by injection or by administering an aerosol, thereby to form a hydrogel at the body temperature of the mammal.

According to a thirty-second aspect of the present invention there is provided a method of temporarily lifting a periodontal ligament tissue and/or supporting periodontal ligament tissue grafting, the method comprising administering to a mammal a composition according to the first aspect of the present invention. In an embodiment, the administration step is made by injection or by administering an aerosol, thereby to form a hydrogel at the body temperature of the mammal.

According to a thirty-third aspect of the present invention there is provided a method of repair and/or restoration of tissue, the method comprising administering to a mammal a hydrogel according to the second aspect of the present invention. In an embodiment, the administration step is made by injection or by administering an aerosol, thereby to form a hydrogel at the body temperature of a mammal.

According to a thirty-fourth aspect of the present invention there is provided a composition according to the first aspect of the present invention for use in the repair and/or restoration of tissue. In an embodiment, the administration step is made by injection or by administering an aerosol, thereby to form a hydrogel at the body temperature of a mammal.

According to a thirty-fifth aspect of the present invention there is provided a composition according to the first aspect of the present invention for use in wound healing. In an embodiment, the administration step is made by injection or by administering an aerosol, thereby to form a hydrogel at the body temperature of a mammal.

According to a thirty-sixth aspect of the present invention there is provided a composition according to the first aspect of the present invention for use in temporary wrinkle reduction. In an embodiment, the administration step is made by injection or by administering an aerosol, thereby to form a hydrogel at the body temperature of a mammal.

According to a thirty-seventh aspect of the present invention there is provided a composition according to the first aspect of the present invention for use in temporarily lifting the base of a scar and promoting healing. In an embodiment, the administration step is made by injection or by administering an aerosol, thereby to form a hydrogel at the body temperature of a mammal.

According to a thirty-eighth aspect of the present invention there is provided a composition according to the first aspect of the present invention for use in supporting dermal connective tissue formation in scar tissue after a surgical intervention and promoting healing. In an embodiment, the administration step is made by injection or by administering an aerosol, thereby to form a hydrogel at the body temperature of a mammal.

According to a thirty-ninth aspect of the present invention there is provided a composition according to the first aspect of the present invention for use in supporting dermal connective tissue formation in scar management of post burn injuries. In an embodiment, the administration step is made by injection or by administering an aerosol, thereby to form a hydrogel at the body temperature of a mammal.

According to a fortieth aspect of the present invention there is provided a composition according to the first aspect of the present invention for use in supporting vascular ingrowth in an acute dermal defect with bleeding and promoting healing. In an embodiment, the administration step is made by injection or by administering an aerosol, thereby to form a hydrogel at the body temperature of a mammal.

According to a forty-first aspect of the present invention there is provided a composition according to the first aspect of the present invention for use in filling a surgically generated dermal cavity. In an embodiment, the administration step is made by injection or by administering an aerosol, thereby to form a hydrogel at the body temperature of a mammal.

According to a forty-second aspect of the present invention there is provided a composition according to the first aspect of the present invention for use in supporting a skin grafting operation. In an embodiment, the administration step is made by injection or by administering an aerosol, thereby to form a hydrogel at the body temperature of a mammal.

According to a forty-third aspect of the present invention there is provided a composition according to the first aspect of the present invention for use in physically delivering bone graft substitutes. In an embodiment, the administration step is made by injection or by administering an aerosol, thereby to form a hydrogel at the body temperature of a mammal.

According to a forty-fourth aspect of the present invention there is provided a composition according to the first aspect of the present invention for use in filling a prosthetic. In an embodiment, the filler has no tissue-inductive properties. In an embodiment, the administration step is made by injection or by administering an aerosol, thereby to form a hydrogel at the body temperature of a mammal.

According to a forty-fifth aspect of the present invention there is provided a composition according to the first aspect of the present invention for use in supporting and repairing periodontal tissue after tooth extraction. In an embodiment, the administration step is made by injection or by administering an aerosol, thereby to form a hydrogel at the body temperature of a mammal.

According to a forty-sixth aspect of the present invention there is provided a composition according to the first aspect of the present invention for use in temporarily lifting a periodontal ligament tissue and/or supporting periodontal ligament tissue grafting. In an embodiment, the administration step is made by injection or by administering an aerosol, thereby to form a hydrogel at the body temperature of a mammal.

According to a forty-seventh aspect of the present invention there is provided a hydrogel according to the second aspect of the present invention for use in repair and/or restoration of tissue. In an embodiment, an administration step is made by injection or by administering an aerosol, thereby to form a hydrogel at the body temperature of a mammal.

In all aspects and embodiments wherein the administration step is made by administering an aerosol, the aerosol may be applied to any opening of the body, such as but not limited to the nasal cavity, the mouth or an open wound.

According to a forty-eighth aspect of the present invention there is provided a kit for forming a hydrogel, the kit comprising: a polymer and a natural or synthetic peptide or protein (NSPP), wherein the polymer comprises: a first monomer for binding water; a second monomer for imparting mechanical properties to said hydrogel; a third monomer for binding to a natural or synthetic peptide or protein (NSPP); and a fourth monomer for imparting phase-transition behaviour; and wherein the natural or synthetic peptide or protein (NSPP) is Thymosin beta-4 or a functional homolog thereof. In an embodiment, the kit further comprises water in a separate container.

According to a forty-ninth aspect of the present invention there is provided a kit for forming a hydrogel, comprising in separate containers: a natural or synthetic peptide or protein (NSPP); and a composition, wherein the composition comprises: a first monomer for binding water; a second monomer for imparting mechanical properties to said hydrogel; a third monomer for binding to a natural or synthetic peptide or protein (NSPP); and a fourth monomer for imparting phase-transition behaviour; wherein the natural or synthetic peptide or protein (NSPP) is Thymosin beta-4 or a functional homolog thereof; and wherein the NSPP and the second monomer are crosslinked, thereby enabling formation of the hydrogel when the composition is contacted with water.

In an embodiment, one or both of the NSPP and the composition are in solid form. In an embodiment, the kit further comprises water in a separate container. In an embodiment, the kit further comprises instructions for the sequential or simultaneous administration of the components of the kit. In an embodiment, the kit is configured such that the composition, the NSPP and water are mixed together when dispensed.

In an embodiment, the NSPP and the composition are used as a filler to deliver bone graft substitutes in situ in a patient in need of treatment with such. The filler keeps BGS in place (adhesive) for at least 6 weeks (degradation) and provides scaffolds for cell ingrowth (bone osteoblasts).

In another embodiment, to form an aerosol for topical administration, the concentration of the polymer in the composition is from about 5 mg/mL to about 70 mg/mL. For instance, the concentration of the polymer in the composition is from about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 mg/mL. The polymer can therefore be delivered via aerosol in very low concentrations, can be adhered to the surgical site and used accordingly.

### DEFINITIONS AND NOMENCLATURE

In describing and claiming the present invention, the following terminology will be used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

As used herein a wording defining the limits of a range or length such as, for example, “from 1 to 5” means any integer from 1 to 5, i.e., 1, 2, 3, 4 and 5. In other words, any range defined by two integers explicitly mentioned is meant to comprise and disclose any integer defining said limits and any integer comprised in said range.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variations found in their respective testing measurements.

The term “room temperature” is intended to mean a temperature of from about 20 to about 25° C.

The term “animal” includes a human and a non-human such as mammal, e.g., a horse, a cow, pig, sheep, cat, dog, etc.

As used herein, an “implant” refers to an article or device that is placed entirely or partially into an animal, for example by a surgical procedure.

As used herein, the term “natural or synthetic peptide or protein” (or NSPP) refers to proteins or peptides that are naturally present in the extracellular part of animal tissue that provides structural support to the animal cells (in addition to performing various other important functions). The term also refers to synthetically prepared proteins or peptides which have analogous function to those naturally-occurring proteins and peptides. By way of example naturally-occurring proteins and peptides are those commonly found in the extracellular matrix (or ECM), which is the defining feature of connective tissue in animals. Naturally-occurring proteins commonly found in the ECM include collagen, fibrin, fibronectin, and laminin (and isoforms thereof).

The NSPP employed in present invention is Thymosin beta-4 or a functional homolog thereof.

The specification uses the following abbreviations:
ECM Extracellular matrix
EHNS N-hydroxyethoxylated succinimide
HEMA Hydroxyethyl methacrylate
LA Lactic acid
NAS N-acryloxysuccinimide
NIPAAm N-isopropylacrylamide
NSPP Natural or synthetic peptide or protein
OEG Oligo(ethylene glycol)
OEGMA Oligo (ethylene) glycol monomethyl ether methacrylate
PBS Phosphate-buffered saline
PEG Polyethylene glycol
PEO Polyethylene oxide
PLA Poly(lactic acid)
PPO Polyethylene oxide-co-propylene oxide
PVA Polyvinyl alcohol
PVP Poly(vinyl pyrrolidone)
PNPHO Poly(N-isopropylacrylamide-co-(N-acryloxysuccinimide)-co-(polylactide/2-hydroxy methacrylate)-co-(oligo(ethylene glycol)/Poly(NIPAAm-co-NAS-co-(HEMA-PLA)-co-OEGMA)
SNHS N-hydroxysulfosuccinimide
TB4 Thymosin beta-4/Thymosin β-4

In an exemplary composition according to the invention, 140 mg/mL PNPHO (ratio 5:8 (5):7:81) is used with 30 mg/mL Thymosin beta-4; this composition was labelled PNPHO-co-TB4 (or alternatively, PNPHO-co-NSPP; or “TR001”) for the purposes of Applicant's clinical trials and is referenced as such throughout the body of this specification.

### BRIEF DESCRIPTION OF THE FIGURES

A preferred embodiment of the invention will now be described, by way of example only, with reference to the accompanying drawings in which:

FIG. 1A is a macroscopic image of the inventive solution (polymer and NSPP) that forms a hydrogel in a simulated physiological condition (PBS at 37° C.) and retains its structure upon gelation.

FIG. 1B is a macroscopic image of PNPHO-co-TB4 injection to a site with active bleeding, showing instant hydrogel formation despite the presence of active bleeding at the defect site.

FIG. 1C is a macroscopic image sequence showing PNPHO-co-TB4 solution and hydrogel formation in contact with a wound at body temperature.

FIG. 1D is a macroscopic image of PNPHO-co-TB4 mixed with blood, forming an adhesive hydrogel which is used to fill a 3D area via layer-by-layer filling.

FIG. 2A shows a synthetic preparation of PNPHO in DMF at 70° C.

FIG. 2B shows the ¹H NMR spectrum of PNPHO in CDCl₃; resonances about 2.9-3.0 ppm for residual trace DMF solvent overlap with those of the NAS protons (e), making original calculations erroneous based on the same data set (integral/area beneath the respective peaks as basis for relative mol % of the respective monomers within the overall PNPHO polymer). Subsequent correction confirms that the third monomer (NAS) is present in an amount of greater than about 7 mol %.

FIG. 3 depicts the solubility of copolymers synthesised at different mole fractions of HEMA-PLA in aqueous solution at 4° C. for lactate number of 3 (a) and 6 (b) (*, **, and *** represent p<0.05, <0.01, and <0.001, respectively).

FIG. 4 shows LCST measurement and comparison between PNPHO-co-TB4 (a) and PNPHO (b). The difference between two LCST values confirms the presence of chemical interaction between two components and as well showing the physical role of TB4 in accelerate the gelation kinetics.

FIG. 5 represents investigation of the scaffolding effect of PNPHO-co-TB4 to integrate with the host tissue. Formation of full-thickness dermal wound (a); use of PNPHO-co-TB4 and Integra for skin grafting (b); and survival of grafts at different time points treated with Integra or PNPHO-co-TB4 (c).

FIG. 6 shows an assessment of inflammatory response to PNPHO-co-TB4 and direct comparison with Integra (dermal matrix gold standard); H&E staining of the Integra treated sites two weeks (a); and four weeks (b) after the surgery; and H&E staining of PNPHO-co-TB4 treated sites after two weeks (c); and four weeks post grafting (d). White arrows show PNPHO-co-TB4 structure and black arrows show fibrous tissue formation around the implants.

FIG. 7 shows an angiogenic response in mice at different time point for PNPHO-co-TB4 and Integra treated sites. Blood vessel formation and ingrowth at the grafted sites were determined by fluorescent radiant efficiency using an IVIS Lumina XR live imager. Two (2) and four (4) weeks post grafting operation, angiogenic responses were determined with an AngioSense750 EX in vivo blood pool fluorescent imaging probe. This near-infrared fluorescent macromolecular probe persists in the vasculature and enables imaging of blood vessels and angiogenesis. At each time point after the surgery, each mouse was injected with 2 nmol AngioSense750EX in 100 μL PBS. After 24 h, each mouse was scanned for fluorescent radiant efficiency (n=8). The radiant efficiency was used to indicate the density of new blood vessels in wound area. Results showed that two weeks post operation, the fluorescent radiant efficiency on PNPHO-co-TB4 treated site was significantly higher (p 0.01) than that of the Integra treated site. In contrast, angiogenic signals were very low for both treatment groups four (4) weeks post operation, indicating that the blood vessel formation was controlled, and the healing of the site was complete.

FIG. 8 shows a histological evaluation of PNPHO-co-TB4 treated sites after 2 weeks post skin grafting operation. Black arrows show blood vessel ingrowth within the structure of PNPHO-co-TB4 hydrogel along with fibroblast infiltration into the injectable scaffolds. Results in (c) and (d) show the formation of blood vessels within the structure of PNPHO-co-TB4. In addition, the staining of the skin biopsies showed clear infiltration of host fibroblast cells within the structure of PNPHO-co-TB4 hydrogels; see, (d).

FIG. 9 shows Masson's trichrome staining of the skin grafted site treated with PNPHO-co-TB4 4 weeks post operation. Black arrows show collagen fibres, deposited from the ingrowth of fibroblast within the structure of PNPHO-co-TB4. To further confirm fibroblast infiltration and skin extracellular matrix formation within the structure of PNPHO-co-TB4, skin grafted sites 4 weeks post grafting were stained with Masson's Trichrome. The results in FIG. 9 show collagen fibre formation within the structure PNPHO-co-TB4 4 weeks post grafting operation. This result confirms the infiltration of fibroblast within the structure of PNPHO-co-TB4 and its potential to integrate with the host tissue and promote neo-dermis formation.

FIG. 10 shows Masson's trichrome staining of the skin grafted site treated with Integra 4 weeks post operation. The formation of collagen fibres within the structure of PNPHO-co-TB4 was significantly higher than that of detected within the structure of Integra. The results showed significantly less collagen formation within the structure of Integra compared with PNPHO-co-TB4.

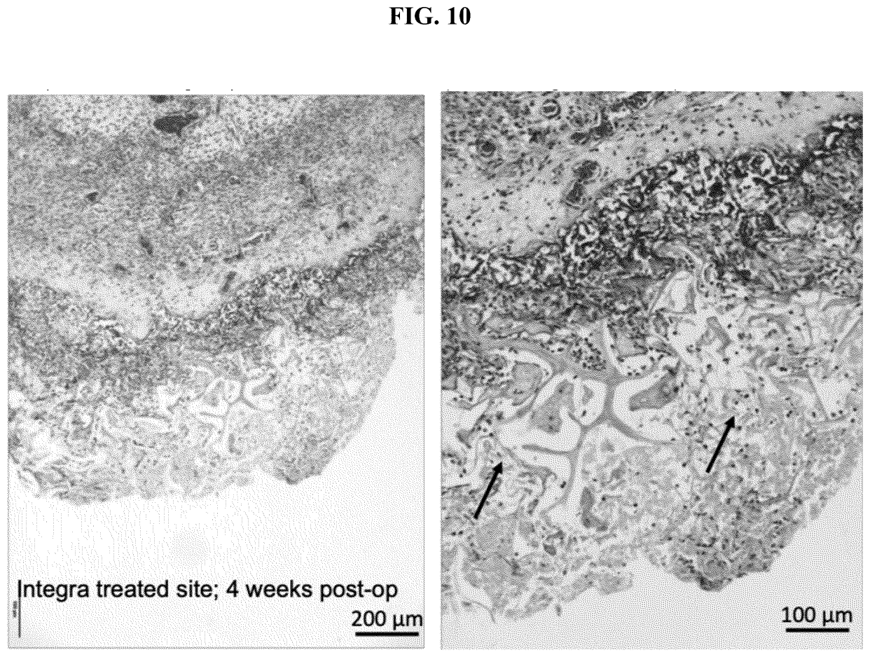

FIG. 11 shows the use of PNPHO-co-TB4 post tooth extraction, (a) extraction site with active bleeding, (b) injection of PNPHO-co-TB4 to the socket site through 21 G needle, (c) instant gelation of PNPHO-co-TB4 at the site and (d) mixture of PNPHO-co-TB4 with blood at the site.

FIG. 12 depicts PNPHO-co-TB4 application post tooth extraction on 10 patients. The clinical use of the device in PET trial showed that PNPHO-co-TB4 injection into the socket site was successful for all ten patients and the principal investigator did not report any device-malfunction.

FIG. 13 shows soft tissue regeneration and wound healing 7 days post operation and treatment with PNPHO-co-TB4. All ten patients treated with PNPHO-co-TB4 returned for the first follow-up visit, one-week post operation. There was no report of pain or discomfort from any of the patients. During the oral examination (one-week post administration), there was no sign of infection or inflammation at the site. In addition, wound closure and soft tissue formation were examined by the Principal Investigator. In all ten patients, wound closure was noted and expedited soft tissue formation was detected.

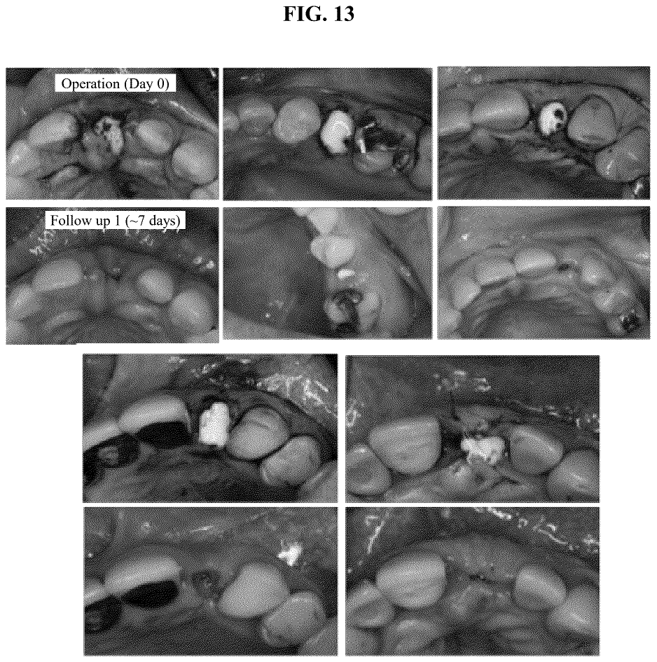

FIG. 14 shows H&E and Masson's trichrome staining of PNPHO-co-TB4 treated samples.

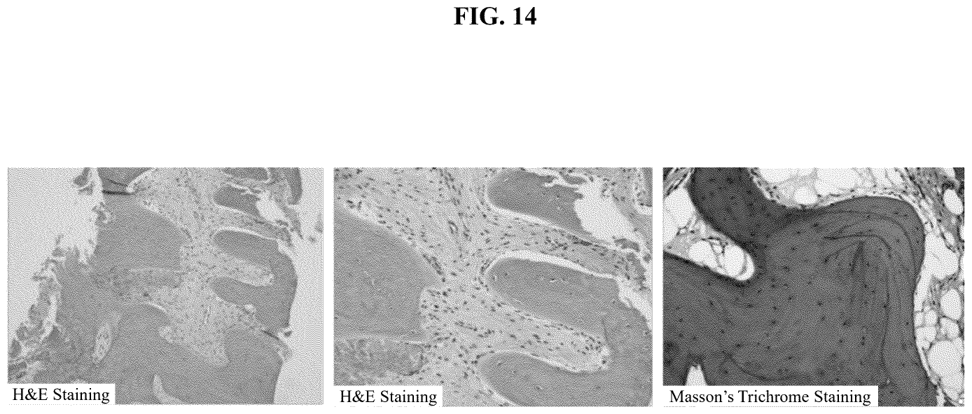

FIG. 15 shows representative images of the plume pattern for the formulations A) 17.5 mg/mL, B) 35 mg/mL and C) 70 mg/mL.

FIG. 16 depicts maximum and minimum plume coverage diameter and derived ovality ratio for the three formulations (average±SD, n=3).

FIG. 17 shows the deposition pattern of the formulations using a human nasal model.

FIG. 18 shows the in vitro release of ciprofloxacin HCl from the PNPHO and PNPHO-co-TB4 hydrogels at 37° C. displayed as cumulative mass (results are indicative, n=1±SD).

### DETAILED DESCRIPTION OF THE INVENTION

The present invention will now be more fully described with reference to the accompanying examples and drawings. It should be understood, however, that the description following is illustrative only and should not be taken in any way as a restriction on the generality of the invention described above.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims. One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

Disclosed herein is the use of a polymer that is tailored for tissue. The compositions of the present invention are preferably injectable.

### 1. Polymers

The term “polymer”, as used herein, refers to a large molecule (macromolecule) composed of repeating structural units (monomers). These subunits are typically connected by covalent chemical bonds. Polymers can be linear or branched polymers. Preferably, the polymers of the present invention are copolymers comprising three or more different monomers.

Accordingly, in one embodiment, preferred polymers used herein include a first water-binding monomer, a second monomer that is capable of imparting mechanical properties to a hydrogel, and a third monomer that has a functional group for binding to an NSPP.

The term “monomer”, as used herein, refers to a structural unit that can be combined to form a polymer, but that itself may also be a polymer, or a derivative of a monomer or polymer. Monomers of this latter type are herein also referred to as “macromonomers”.

Herein a “macromonomer” is a polymer or oligomer the molecules of which each have one end-group that acts as a monomer molecule, so that each polymer or oligomer molecule contributes only a single monomer unit to a chain of the product polymer.

The polymer of the compositions of the present invention comprises: a first monomer for binding water; a second monomer for imparting mechanical properties to said hydrogel; a third monomer for binding to a natural or synthetic peptide or protein (NSPP); and a fourth monomer for imparting phase-transition behaviour.

### 1.1. First Monomer: Water-Binding Monomer

As discussed above, the advantageous properties of the preferred hydrogels used herein can be attributed to the combination of an NSPP and the particular components of the preferred polymers. One particular advantageous property of these preferred polymers is their water-binding capacity. The presence of water in the hydrogels provides both an environment that resembles the natural environment of the damaged tissue (which assists in tissue regeneration), and the required compression resistance to the hydrogel.

Accordingly, the preferred polymers used herein should include monomers or units that are able to bind water to such a capacity that a hydrogel is able to form when the polymer is contacted with an NSPP and water. In addition, the hydrogel thus formed should have the required compression resistance and resilience.

A person skilled in the art will understand that water-binding monomers need to be present in the preferred polymers used in the present invention in proportions that are sufficient to produce a polymer that fulfils these requirements. Generally, the proportion of water binding monomers in the polymer may be: about 10:1, about 5:1, about 4:1, about 3:1, about 2:1, about 1:1, about 1:2, about 1:3, about 1:4, or about 1:5 in a molar ratio of water binding:mechanical strength monomers. In fact, the water-binding monomers need to make the polymer not only hydrophilic but impart much more significant water-binding capacities to the polymer.

Accordingly, preferred polymers to be used in the present invention will have water-binding capacities of between about 70% and about 500%, between about 80% and about 400%, between about 90% and 300% or between about 100% and 200%. For example, the water-binding capacity of the preferred polymers used herein is about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500%.

Suitable examples of water-binding monomers include those that can be synthesised into polymers such as polyethers (e.g., alkaline polyimides such as polyethylene glycol (PEG), oligo(ethylene glycol) (OEG), polyethylene oxide (PEO), polyethylene oxide-co-propylene oxide (PPO), co-polyethylene oxide block or random copolymers, polyvinyl alcohol (PVA), poly(vinyl pyrrolidone) (PVP), poly(amino acids) and dextran. The polyethers, and more particularly oligo(oxyalkylenes) (e.g., OEG), are especially preferred, because they have the requisite water-binding capacity, are simple to synthesise and/or purchase, and are inert, in the sense that they illicit minimal or no immune response from the tissues into which they are placed.

In addition, any of a variety of hydrophilic functionalities can be used to make a monomer (and therefore a polymer formed from such a monomer) water soluble. For example, functional groups like phosphate, sulphate, quaternary amine, hydroxyl, amine, sulfonate and carboxylate, which are water soluble, may be incorporated into a monomer to make it water soluble.

Monomers may also be reacted with other compounds to form “macromonomers”. Thus, the first monomer may optionally be a macromonomer.

A preferred first monomer which is a macromonomer is oligo(ethyleneglycol) monomethyl ether methacrylate (OEGMA), which is a hydrophilic monomer composed of two hydrophilic monomers: ethylene glycol and methacrylate.

Preferably, the polymer comprises the first monomer in an amount of from about 3 to about 8 mol %, preferably, about 3, 4, 5, 6, 7 or 8 mol %.

### 1.2. Second Monomer: Monomer Imparting Mechanical Properties

As discussed above, the advantageous properties of the preferred hydrogels used with the present invention can be attributed, in part, to the particular components that make up the polymers. In some embodiments, the preferred polymers used in the present invention are able to contribute additional mechanical properties to the hydrogels.

A person skilled in the art will understand that monomers capable of imparting mechanical properties to a hydrogel need to be present in the preferred polymers in proportions that are sufficient to produce a hydrogel having the desired mechanical properties. Generally, the proportion of “mechanical” monomers in the polymer may be: about 10:1, about 5:1, about 4:1, about 3:1, about 2:1, about 1:1, about 1:2, about 1:3, about 1:4, or about 1:5 in a molar ratio of water binding:mechanical strength monomers. Suitable examples of monomers that are capable of imparting mechanical properties (e.g., compression resistance) to a hydrogel include methacrylates such as hydroxyethyl methacrylate (HEMA), a hydroxyethyl methacrylate poly(lactic acid) copolymer (HEMA-PLA), polyesters such as poly(lactic acid), poly(caprolactone), poly(glycolide), and their random co-polymers (e.g., poly(glycolide-co-lactide) and poly(glycolide-co-caprolactone)).

Monomers may also be reacted with other compounds to form “macromonomers”. A preferred second monomer which is a macromonomer is hydroxyethyl methacrylate poly(lactic acid) (HEMA-PLA).

Preferably, the polymer comprises the second monomer in an amount of from about 5 to about 9 mol %, preferably, about 5, 6, 7, 8 or 9 mol %.

### 1.3. Third Monomer: NSPP-Binding Monomer

As discussed above, the preferred hydrogels used in the present invention form by combining the polymer with an NSPP, in the presence of water. In order to effectively combine the polymer with the NSPP, preferably monomers or units that have a crosslinking ability are included in the polymer.

This crosslinking ability means that the polymers are able to bind to NSPPs (as discussed further below) and, by doing so, crosslink the NSPP to form hydrogels containing the NSPP. Alternatively, via a similar mechanism, the NSPPs act as the crosslinker, thereby crosslinking the polymer to form a hydrogel.

By utilising a polymer design in which a monomer having a functional group for binding with Thymosin beta-4 or the like is provided in the polymer, the inventors have recognised that polymers do not need to be further crosslinked with, for example, chemical or UV crosslinking, to form a hydrogel.

In addition, by covalently binding the NSPP to the polymer, the NSPP is more effectively retained in the hydrogel network, which means that, once the hydrogel is administered to the repair site, the NSPP is not able to migrate easily away from the site. This means that the structural integrity of the gel at the repair site is maintained (due to the mechanical properties of NSPPs, as mentioned above), and assists in providing an environment at the repair site that closely mimics the natural environment of the tissue.

In order to produce a polymer that is capable of binding to an NSPP, a person skilled in the art will understand that monomers capable of binding to an NSPP need to be present in the polymers of the present invention in proportions that are sufficient to crosslink with an NSPP, such that a hydrogel can be formed in the presence of water. Generally, the proportion of “crosslinking” monomers in the polymer is at least about 1:1 molar ratio of crosslinking monomer:water binding monomer. This ratio can increase to, for example, about 2:1, about 3:1, about 4:1, about 5:1, about 6:1, about 7:1, about 8:1, about 9:1 or about 10:1.

Monomers that are capable of binding to NSPPs generally have either electrophilic or nucleophilic functional groups, such that a nucleophilic functional group on, for example, an NSPP may react with an electrophilic functional group on the monomer, to form a covalent bond. Preferably, the polymer comprises more than two NSPP-binding monomers, so that, as a result of electrophilic-nucleophilic reactions, the polymer combines with the NSPP to form crosslinked polymeric products. Such reactions are referred to as “crosslinking reactions”.

Therefore, for example, if an NSPP has nucleophilic functional groups such as amines, the polymer may have electrophilic functional groups such as N-hydroxysuccinimides (NHS). Other electrophilic functional groups that are suitable for use in the present invention are N-hydroxysulfosuccinimide (SNHS) and N-hydroxyethoxylated succinimide (ENHS). An example of a monomer of this type is N-acryloxysuccinimide (NAS). On the other hand, if an NSPP has electrophilic functional groups, then the polymer may have nucleophilic functional groups such as amines or thiols.

Preferably, the polymer comprises the third monomer in an amount of at least about 7 mol %, more preferably at least about 7, 8, 9, 10, 11, 12, 13, 14, 15, 16, 17, 18 or 19 mol %.

### 1.4. Fourth Monomer: Phase-Transition Monomer

In another embodiment, the preferred polymer may further include a fourth monomer that is capable of imparting phase transition characteristics to the hydrogel, thereby enabling the composition to be in an injectable form at room temperature, and to enable gel formation (i.e., hydrogel formation) at body temperature. Further, these phase transition characteristics allow the preferred polymers used with the present invention to form hydrogels, of which various properties (such as viscosity) can be varied by altering factors such as pH and temperature.

Thermoresponsive injectable hydrogels are designed such that the lower critical solution temperature (LCST) is below body temperature. Therefore, gelation can be achieved simply by increasing the temperature of the hydrogel by, for example, letting it warm up to body temperature (which occurs when the hydrogel is administered into the body). Various thermoresponsive and injectable polymers including poly(ethylene oxide)/poly(propylene oxide) and poly(N-isopropylacrylamide) (PNIPAAm) homopolymers and copolymers are suitable for use in the present invention. NIPAAm (as a monomer building block or poly NIPAAm) is particularly suitable, as it has a LCST of 32° C., allowing it to be in the gel form at body temperature.

In order to produce a polymer that is thermoresponsive, a person skilled in the art will understand that the phase-transition monomers need to be present in the polymers used with the present invention in proportions that are sufficient to enable the viscosity of a hydrogel including the polymer to be varied by exposure of the hydrogel to different conditions of temperature and pH. Generally, the proportion of “phase-transition” monomers in the polymer is at least about 9:1 molar ratio of phase-transition monomer:water binding monomer. This ratio can increase to, for example: about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, or about 30:1 in a molar ratio of phase-transition monomer:water binding monomer.

The viscosity of the preferred hydrogels used with the present invention, at lower temperatures (e.g., 4° C.), is such that the hydrogel is injectable. The hydrogel then becomes more viscous as the temperature increases, forming a gel having the desired viscosity at a temperature of about 37° C. This means that the preferred hydrogel used with the present invention, at cooler temperatures, can be administered easily to the site of repair by, for example, injection or administration by aerosol. The hydrogel is then transformed, by warming in the body to the body's natural temperature, into a more viscous gel, which has the desired strength and elasticity properties.

Preferably, the polymer comprises the fourth monomer in an amount which makes up the remainder to 100% of the polymer composition. In an embodiment, the mol % of the fourth monomer can be up to about 85%, preferably, about 64, 65, 66, 67, 68, 69, 70, 71, 72, 73, 74, 75, 76, 77, 78, 79, 80, 81, 82, 83, 84 or 85 mol %.

### 1.5. Other Polymer Properties

It will be understood by a person skilled in the art that, by combining different types of monomers, polymers can be produced that have a range of different properties. In addition, by incorporating particular monomers or functional groups into a pre-existing polymer, the properties of the polymer can be modified. For example, co-polymerisation of HEMA monomers with other monomers (such as methyl methacrylate) can be used to modify properties such as swelling and mechanical properties. Monomers may also be reacted with other compounds to form “macromonomers” (mentioned above) that are then included in the preferred polymers used in the present invention. For example, HEMA can be reacted with lactide to form a HEMA-poly-lactic acid polymer (HEMA-PLA), which itself can be used as a monomer in the polymers of the present invention. In addition, the monomers themselves may be combinations of monomer units, which are then incorporated into the polymer. An example of this type of monomer is oligo(ethyleneglycol) monomethyl ether methacrylate (OEGMA), which is a hydrophilic monomer composed of two hydrophilic monomers: ethylene glycol and methacrylate.

The preferred polymers used in the present invention may be further modified with one or more moieties and/or functional groups. Any moiety or functional group can be used as required. In some embodiments, polymers may be modified with polyethylene glycol (PEG), with a carbohydrate, and/or with acyclic polyacetals derived from polysaccharides. In addition, as discussed above, hydrophilic groups can be incorporated into monomers (and therefore polymers) to increase a polymer's water-binding capacity.

In terms of sequence, copolymers may be block copolymers, graft copolymers, random copolymers, blends, mixtures, and/or adducts of any of the foregoing and other polymers. Typically, polymers used in accordance with the present invention are organic polymers. Preferably, the polymers used in the present invention are biocompatible. In some embodiments, the polymers are biodegradable. In other embodiments, the polymers are both biocompatible and biodegradable.

The preferred polymers used in the present invention may also include other monomers in their structure. For example, the monomers may be polymers such as poly(vinyl alcohol) (PVA), polyesters, acrylic polymers and ionic polymers, or monomers of these.

If it is desired that the polymer be biodegradable or absorbable, one or more monomers having biodegradable linkages may be used. In the alternative, or in addition, the monomers may be chosen such that the product of the reaction between them results in a biodegradable linkage. For each approach, monomers and/or linkages may be chosen such that the resulting biodegradable polymer will degrade or be absorbed in a desired period of time, e.g., from about 6 h to about 6 months. Preferably, the monomers and/or linkages are selected such that, when the polymer degrades under physiological conditions, the resulting products are nontoxic.

The biodegradable linkage may be chemically or enzymatically hydrolysable or absorbable. Illustrative, exemplary and non-limiting chemically-hydrolysable biodegradable linkages include polymers, copolymers and oligomers of glycolide, lactide, caprolactone, dioxanone, and trimethylene carbonate. Illustrative enzymatically-hydrolysable biodegradable linkages include peptidic linkages cleavable by metalloproteinases and collagenases. Additional illustrative biodegradable linkages include polymers and copolymers of poly(hydroxyl acid)s, poly(orthocarbonate)s, poly(anhydride)s, poly(lactone)s, poly(aminoacid)s, poly(carbonate)s, and poly(phosphonate)s.

The chemical hydrolysation of lactide in the invention results in the increase of lower critical solution temperature (LCST) of the polymer (by decreasing the overall hydrophobicity of the polymer) and thus its bioresorptive capacity.

### 1.6. Preferred Polymers

The polymer preferably comprises the first monomer in an amount of from about 3 to about 8 mol %, such as from about 4 to about 6 mol % or about 4, 5, 6 mol %.

The polymer preferably comprises the second monomer in an amount of from about 5 to about 9 mol %, such as from about 6 to about 8 mol % or about 6, 7 or 8 mol %.

The polymer preferably comprises the third monomer in an amount of at least about 7 mol %, such as about 7, 8, 9, 10, 11, 12, 13, 14, 15, 16, 17, 18 or 19 mol %.

The polymer preferably comprises the fourth monomer in an amount which makes up the remainder to 100% of the polymer composition, for example, from about 60 and about 81 mol %, such as about 64, 65, 66, 67, 68, 69, 70, 71, 72, 73, 74, 75, 76, 77, 78, 79, 80, 81, 82, 83, 84, or 85 mol %.

The percentages recited herein relate to the composition of the final polymer and not the feed amounts utilised when forming the polymer.

In one embodiment, the polymer preferably comprises:
i. the first monomer in an amount of from about 3 to about 8 mol % (for example from about 4 to about 6 mol %);
ii. the second monomer in an amount of from about 5 to about 9 mol % (for example from about 6 to about 8 mol %);
iii. the third monomer in an amount of at least about 7 mol %; and
iv. the fourth monomer in an amount of up to about 85 mol % (for example up to about 81 mol %).

In another embodiment, the polymer preferably comprises:
i. the first monomer in an amount of about 5 mol %;
ii. the second monomer in an amount of about 7 mol %;
iii. the third monomer in an amount of about 7 mol %; and
iv. the fourth monomer in an amount of about 81 mol %.

In one embodiment, the preferred polymer used in the present invention is a polymer of Formula (I):

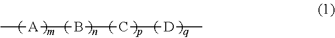

wherein
A is the first monomer (a water-binding monomer);
B is the second monomer (a monomer that is capable of imparting mechanical properties to a hydrogel);
C is the third monomer (a monomer that has a functional group for binding to an NSPP);
D is the fourth monomer (a monomer that is capable of imparting phase transition characteristics to the hydrogel);
m is an integer from 1 to 20; for example, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2;
n is an integer from 1 to 20; for example, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2;
p is an integer from 1 to 20; for example, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2; and
q is an integer from 1 to 20; for example, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2.

Preferably, the ratio of m:n:p:q is about 5:8(5):7:81. A person skilled in the art will be aware that the monomers A, B, C and D may be present in the polymer in any order, provided that the required water-binding, strengthening and/or cross-linking capabilities are achieved.

A, B, C and D may preferably be present in the mol % ranges provided above in the context of the first, second, third and fourth monomers, respectively.

An example of a polymer of Formula (I) is a polymer of Formula (Ia):

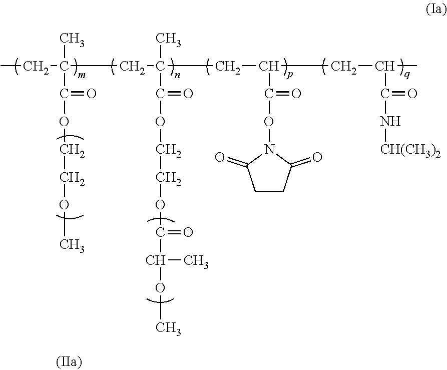

wherein A is the water-binding monomer OEGMA, B is the strengthening monomer HEMA-PLA, C is the crosslinker NAS, D is the phase-transition monomer NIPAAm, and m, n, p, q, x and y are as defined above.

A person skilled in the art will be aware that the monomers A, B, C and D may be present in the polymer in any order, provided that the required water-binding, strengthening and/or cross-linking capabilities are achieved.

It has also been discovered that some monomers, such as HEMA-PLA, polyesters such as poly(lactic acid), poly(caprolactone), poly(glycolide), and their random copolymers (e.g., poly(glycolide-co-lactide) and poly(glycolide-co-caprolactone) and other biodegradable and biocompatible polymers, can elevate the LCST of the preferred polymer used in the present invention during degradation of biodegradable segments (e.g., PLA) in vivo, leading to bioresorption of the polymer. This provides the additional advantage that the polymers used in the present invention may be designed so as to be biodegradable in vivo.

The overall size of the preferred polymer used in the present invention may differ, depending on factors such as the types of monomers that are incorporated into the polymer, the type of NSPP that is sought to be used to form the hydrogel, and the conditions under which the protein is to be coupled to the polymer. However, in general, the preferred polymer used in the present invention may be a molecule of about 1 to about 100 kDa, about 5 to about 60 kDa, or about 30 kDa.

### 1.7. PNPHO

A preferred polymer is PHPHO. The polymer PNPHO preferably comprises OEGMA in an amount of from about 3 and about 8 mol %, such as from about 4 and about 7 mol % or about 3 4, 5, 6, 7 or 8 mol %.

The polymer preferably comprises HEMA-PLA in an amount of from about 5 and about 9 mol %, such as from about 6 and about 8 mol % or about 3, 4, 5, 6, 7 or 8 mol %. The polymer preferably comprises NAS in an amount of at least about 7 mol %, such as about 7, 8, 9, 10, 11, 12, 13, 14, 15, 16, 17, 18 or 19 mol %.

The polymer preferably comprises NIPAAm in an amount which makes up the remainder to 100% of the polymer composition, for example, from about 64 to about 85 mol %, such as about 64, 65, 66, 67, 68, 69, 70, 71, 72, 73, 74, 75, 76, 77, 78, 79, 80, 81, 82, 83, 84, or 85 mol %.

The percentages recited herein relate to the composition of the final polymer and not the feed amounts utilised when forming the polymer.

In one embodiment, preferably the polymer comprises:
i. OEGMA in an amount of from about 3 to about 8 mol % (for example from about 4 to about 6 mol %);
ii. HEMA-PLA in an amount of from about 5 to about 9 mol % (for example from about 6 to about 8 mol %);
iii. NAS in an amount of at least about 7 mol %; and
iv. NIPAAm in an amount of up to about 85 mol % (for example up to about 81 mol %).

In another embodiment, the polymer comprises:
i. OEGMA in an amount of about 5 mol %;
ii. HEMA-PLA in an amount of about 7 mol %;
iii. NAS in an amount of about 7 mol %; and
iv. NIPAAm in an amount of about 81 mol %.

A preferred form of the polymer PNPHO for use in the present application is a polymer of Formula (Ia), as drawn above.

Based on Formula I, defined previously:
i. A is oligo(ethylene) glycol monomethyl ether methacrylate OEGMA;
ii. B is hydroxyethyl methacrylate poly(lactic acid) (HEMA-PLA);
iii. C is N-acryloxysuccinimide (NAS); and
iv. D is N-isopropylacrylamide (NIPAAm).

In addition, x is in the range of 1-1000 and y is in the range of 1-1000 and m, n, p, and q are in the range of 1-20.

A person skilled in the art will be aware that the monomers A, B, C and D may be present in the polymer in any order, provided that the required water-binding, strengthening and/or cross-linking capabilities are achieved.

### 1.8. Synthesis of Polymers

A person skilled in the art will be aware of suitable methods of synthesising the preferred polymers used in the present invention. These include methods such as ring-opening polymerisation, addition polymerisation (including free radical polymerisation) and condensation polymerisation.

Formation of the preferred polymer, PNPHO, is described in the examples below.

### 2. Compositions for Forming Hydrogels

The present invention also relates to a preferred composition useful for forming a hydrogel for use in the invention.

The composition of the present invention comprises a polymer and an NSPP, the polymer comprising:
i. a first water-binding monomer; and
ii. a second monomer that imparts mechanical properties;
iii. a third monomer that is an NSPP-binding monomer, comprising a functional group that is capable of binding to the NSPP;
iv. a fourth monomer capable of imparting phase transition characteristics to the hydrogel;
wherein the natural or synthetic peptide or protein (NSPP) is Thymosin beta-4 or a functional homolog thereof;
and wherein the binding of the NSPP to the second monomer crosslinks the polymer, thereby enabling formation of a hydrogel when the composition is contacted with water.

The term “composition”, as used herein, refers to a solid or liquid composition containing the components mentioned above. In some embodiments, other components such as pharmaceutically-acceptable excipients and biologically active agents (e.g., drugs, vitamins and minerals), to assist in repair and/or re-generation of the target bone tissue, and/or to provide a method of achieving targeted delivery of biologically active compounds, may also be included in the preferred compositions used in the present invention.

In general, the amount of polymer in the composition used in the present invention is an amount that allows for the formation of hydrogels.

In some embodiments, the amount of polymer in the composition ranges: from about 1% w/w to about 90% w/w, from about 2% w/w to about 80% w/w, from about 4% w/w to about 70% w/w, from about 5% w/w from about 60% w/w, from about 5% w/w to about 50% w/w, from about 6% w/w to about 40% w/w, from about 7% w/w to about 30% w/w or from about 8% w/w to about 20% w/w.

In some embodiments, the amount of polymer is: about 1% w/w, about 2% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w or more. In some embodiments, the amount of polymer is approximately 85% w/w.

As a general rule, the solidity of the hydrogel increases with higher polymer concentrations in the composition.

In general, the amount of NSPP in the composition of the present invention is an amount that allows for the formation of hydrogels.

In some embodiments, the amount of NSPP in the composition ranges: from about 0.01% w/w to about 60% w/w, from about 1% w/w to about 50% w/w, from about 1% w/w to about 40% w/w, from about 5% w/w to about 30% w/w, from about 5% w/w to about 20% w/w, or from about 5% w/w to about 10% w/w.

In some embodiments, the percent of NSPP is about 1% w/w, about 2% w/w, about 3% w/w, about 4% w/w, about 5% w/w, about 6% w/w, about 7% w/w, about 8% w/w, about 9% w/w, about 10% w/w, about 20% w/w, about 30% w/w, about 40% w/w, about 50% w/w, or more.

The % w/w is based on the total weight of the composition before the composition is contacted with water.

In one embodiment, the composition comprises equimolar amounts of the polymer and Thymosin beta-4 or a functional homolog thereof.

### 2.1. Excipients and Biologically-Active Agents

Pharmaceutically-acceptable excipients may be included in the preferred compositions and/or hydrogels used in the present invention, and include any and all solvents, dispersion media, inert diluents, or other liquid vehicles, dispersion or suspension aids, granulating agents, surface active agents, disintegrating agents, isotonic agents, thickening or emulsifying agents, preservatives, binding agents, lubricants, buffering agents, oils, and the like, as suited to the particular dosage form desired. Remington (Gennaro, A. R., Remington: The Science and Practice of Pharmacy, 21st Ed (2006) Lippincott Williams & Wilkins) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

Excipients such as colouring agents, coating agents, sweetening, flavouring, and perfuming agents can be present in the composition, according to the judgment of the formulator.

Biologically active agents or drug compounds that may be added to the preferred composition and/or hydrogel used in the present invention include proteins, glycosaminoglycans, carbohydrates, nucleic acids and inorganic and organic biologically active compounds, such as enzymes, antibiotics, anti-neoplastic agents, local anaesthetics, hormones, angiogenic agents, anti-angiogenic agents, growth factors (e.g., insulin-like growth factor-1 (IGF-1), basic fibroblast growth factor (bFGF) and transforming growth factor-b (TGFb), antibodies, neurotransmitters, psychoactive drugs, anticancer drugs, chemotherapeutic drugs, drugs affecting reproductive organs, genes, and oligonucleotides.

A composition containing components such excipients and/or biologically active agents can be produced by combining a preferred polymer as disclosed herein with an NSPP, drying the resulting composition, and then combining this with one or more other components. The resulting composition may be in the form of a powder or other particulate form, to which water is then added to form a hydrogel, in accordance with the present invention. A hydrogel containing these components can therefore be produced simply by adding the desired aqueous solvent to the composition.

The amount of polymer, NSPP and biologically active agent present in the preferred composition to be used in the invention will necessarily depend upon the particular drug and the condition to be treated. A person skilled in the art will be aware of appropriate agents and amounts to use to treat the condition.

Exemplary embodiments include fat grafts, demineralised bone matrices (DBM), autologous grafts (i.e., biologically active grafts). The role of this class of additives is to impart tissue inductive properties to the composite. For instance, there are low concentrations of growth factors in DBM/autologous grafts, etc.

Further exemplary embodiments include bone particles (from human or animals) as well as purely inactive space fillers such as glass beads. The role of this class of additives is to provide the required 3D structure to the composite.

It will be appreciated by one of ordinary skill in the art that by bone graft substitutes is meant a broad range of particles including but not limited to synthetic calcium/phosphate particles, animal derived bone particles and non-processed human bones.

### 2.2. Natural or Synthetic Peptide or Protein (NSPP)

In the context of the present invention, an NSPP is relevant because, as discussed above, it crosslinks polymers, which enables the polymers to form a hydrogel. The preferred hydrogels used in the present invention may be formed by, for example, exposing Thymosin beta-4 to a polymer of Formula (I). The NSPP is also important because it provides additional mechanical properties (such as strength and resilience) to the hydrogel, as well as providing, at the repair site, an environment that mimics the natural environment, thereby assisting in tissue repair and re-generation.

It is important that the NSPP contains side chains or other functional groups that are exposed to enable reaction with the functional group of the NSPP-binding monomer(s), thereby binding the NSPP to the polymer through the NSPP-binding monomer(s). Examples of suitable side chains include glutamic acid or lysyl side chains.

The present invention also contemplates the use of variants of the NSPPs, for example species variants or polymorphic variants. The present invention is intended to cover all functionally active variants of the NSPPs that exhibit the same activity. This also includes apo- and haloforms of the NSPPs, post-translationally modified forms, as well as glycosylated or deglycosylated derivatives. Such functionally active fragments and variants include, for example, those having conservative amino acid substitutions.

Preferably, the NSPP(s) for use in the present invention will be obtained from recombinant sources, although they can also be extracted from natural sources or synthesised.

### 2.3 Thymosin Beta-4

A preferred NSPP is Thymosin beta-4. Thymosin beta-4 is a highly conserved, naturally occurring, water-soluble regenerative peptide that is found in all tissues and in all cell types, except red blood cells. It is also found in the blood and in other body fluids, including tears, saliva, cerebrospinal fluid, and wound fluids.

Human Thymosin beta-4 has the following sequence: SDKPDMAEIE KFDKSKLKKT ETQEKNPLPS KETIEQEKQA GES.

Thymosin beta-4 is alternatively written as: Ser-Asp-Lys-Pro-Asp-Met-Ala-Glu-Ile-Glu-Lys-Phe-Asp-Lys-Ser-Lys-Leu-Lys-Lys-Thr-Glu-Thr-Gln-Glu-Lys-Asn-Pro-Leu-Pro-Ser-Lys-Glu-Thr-Ile-Glu-Gln-Glu-Lys-Gln-Ala-Gly-Glu-Ser.

For the purposes of the ensuing discussion, the terms Thymosin beta-4 and TB4 are used synonymously; TB4 being a shorthand notation for Thymosin beta-4.

According to an exemplary embodiment of the invention, Thymosin beta-4 is most preferably used in an approximate 1:1 molar ratio with the PHPHO. However, this can be toggled depending on intended application. In an exemplary composition, 140 mg/mL PNPHO (ratio 5:8(5):7:81) is used with 30 mg/mL Thymosin beta-4; this composition was labelled “PNPHO-co-TB4” for the purposes of Applicant's clinical trials.

### 2.4 Functional Homologs (Isoforms) of Thymosin Beta-4

An alternative preferred NSPP is a functional homolog of Thymosin beta-4. Functional homologs of the polypeptides described above are also suitable for use in the compositions and methods described herein. A functional homolog is a polypeptide that has sequence similarity to a reference polypeptide, and that carries out one or more of the biochemical or physiological function(s) of the reference polypeptide.

A functional homolog and the reference polypeptide may be natural occurring polypeptides, and the sequence similarity may be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, may themselves be functional homologs.

Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide: polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides. Sequence analysis can involve BLAST, Reciprocal BLAST, or PSI-BLAST analysis of nonredundant databases using an amino acid sequence as the reference sequence. Amino acid sequence is, in some instances, deduced from the nucleotide sequence. Those polypeptides in the database that have greater than 40% sequence identity are candidates for further evaluation for suitability as a polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in polypeptides, e.g., conserved functional domains.

Conserved regions can be identified by locating a region within the primary amino acid sequence of a polypeptide that is a repeated sequence, forms some secondary structure (e.g., helices and beta sheets), establishes positively or negatively charged domains, or represents a protein motif or domain; see, e.g., the Pfam website describing consensus sequences for a variety of protein motifs and domains at www.sanger.ac.uk/Software/Pfam/and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

### 3. Hydrogels

The present invention also relates to a hydrogel comprising a polymer according to the invention, an NSPP and water, wherein the polymer comprises:
i. a first water-binding monomer;
ii. a second monomer that imparts mechanical properties;
iii. a third monomer that is an NSPP-binding monomer, comprising a functional group that is capable of binding to the NSPP;
iv. a fourth monomer capable of imparting phase transition characteristics to the hydrogel;
wherein the natural or synthetic peptide or protein (NSPP) is Thymosin beta-4 or a functional homolog thereof;
and wherein the binding of the NSPP to the third monomer crosslinks the polymer, thereby forming a hydrogel, with the water contained therein.

In one embodiment, the hydrogel includes a polymer having a monomer described above for enabling phase transition of the hydrogel from liquid state at lower temperature to gel state at body temperature. One example of a monomer useful for this purpose is NIPAAm. Thymosin beta-4, can be made to transition from liquid to gel state according to temperature profile by use of this monomer. Therefore, the advantage is that the preferred hydrogel used in the present invention, at cooler temperatures, can be administered easily by, for example, injection or aerosol. The hydrogel is then transformed into a more viscous gel, which has the desired strength and elasticity properties, following warming in the body to the natural body temperature.

Having been provided with a polymer composition described above, the hydrogel may be formed by adding water to the composition in any way known to a person skilled in the art. Indeed, one advantage of the present invention is that the polymer does not need to be crosslinked in any way prior to contact with the NSPP, in order for a hydrogel to form.

### 3.1. Cells

The preferred hydrogel for use in the present invention may also include cells to assist in repair and/or re-generation of the target tissue.

In general, cells to be used in accordance with the present invention are any types of cells. The cells should be viable when encapsulated or immobilised within the preferred hydrogels used in the present invention. The product of the invention is effective in immobilising the cells. For instance, a layer of hydrogel injected/sprayed at a site, with cells subsequently added to the sticky hydrogel and then covered with another layer of the hydrogel achieve the sought result. As such, the encapsulation happens inside the body, not outside.

In some embodiments, cells that can be encapsulated within hydrogels include, but are not limited to: mammalian cells (e.g., human cells, primate cells, mammalian cells, rodent cells, etc.), avian cells, fish cells, insect cells, plant cells, fungal cells, bacterial cells, and hybrid cells. In some embodiments, exemplary cells that can be encapsulated within hydrogels include stem cells, totipotent cells, pluripotent cells, and/or embryonic stem cells.

In some embodiments, exemplary cells that can be encapsulated/immobilised within hydrogels include, but are not limited to, primary cells and/or cell lines from any tissue. For example: cardiomyocytes, myocytes, hepatocytes, keratinocytes, melanocytes, neurons, astrocytes, embryonic stem cells, adult stem cells, hematopoietic stem cells, hematopoietic cells (e.g., monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc., and/or hybrids thereof, may be encapsulated within preferred hydrogels used in accordance with the present invention.

Exemplary mammalian cells that can be encapsulated within the preferred hydrogels used in accordance with the present invention include, but are not limited to: Chinese hamster ovary (CHO) cells, Hela cells, Madin-Darby canine kidney (MOCK) cells, baby hamster kidney (BHK cells), NSO cells, MCF-7 cells, MDA-MB-438 cells, U87 cells, A172 cells, HL60 cells, A549 cells, SP10 cells, DOX cells, DG44 cells, HEK 293 cells, SHSY5Y, Jurkat cells, BCP-1 cells, COS cells, Vero cells, GH3 cells, 9L cells, 3T3 cells, MC3T3 cells, C3H-10T1/2 cells, NIH-3T3 cells, and C6/36 cells.

In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, cells are layered within a hydrogel. In some embodiments, the hydrogel contains different cell types.

In some embodiments, the conditions under which cells are encapsulated within hydrogels are altered in order to maximise cell viability. In some embodiments, for example, cell viability increases with lower polymer concentrations. In some embodiments, cells located at the periphery of a hydrogel tend to have decreased viability relative to cells that are fully-encapsulated within the hydrogel. In some embodiments, conditions (e.g., pH, ionic strength, nutrient availability, temperature, oxygen availability, osmolarity, etc.) of the surrounding environment may need to be regulated and/or altered to maximise cell viability.

In some embodiments, cell viability can be measured by monitoring one of many indicators of cell viability. In some embodiments, indicators of cell viability include, but are not limited to: intracellular esterase activity, plasma membrane integrity, metabolic activity, gene expression, and protein expression. To give but one example, when cells are exposed to a fluorogenic esterase substrate (e.g., calcein AM), live cells fluoresce green as a result of intracellular esterase activity that hydrolyses the esterase substrate to a green fluorescent product. To give another example, when cells are exposed to a fluorescent nucleic acid stain (e.g., ethidium homodimer-1), dead cells fluoresce red because their plasma membranes are compromised and, therefore, permeable to the high-affinity nucleic acid stain.

In general, the number/amount of cells in a composition is an amount that allows for the formation of preferred hydrogels for use in accordance with the present invention. In some embodiments, the amount of cells that is suitable for forming hydrogels ranges: from about 0.1% w/w to about 80% w/w, from about 1.0% w/w to about 50% w/w, from about 1.0% w/w to about 40% w/w, from about 1.0% w/w to about 30% w/w, from about 1.0% w/w to about 20% w/w, from about 1.0% w/w to about 10% w/w, from about 5.0% w/w to about 20% w/w, or from about 5.0% w/w to about 10% w/w. In some embodiments, the number/amount of cells in a composition that is suitable for forming hydrogels is approximately 5% w/w.

In some embodiments, the concentration of cells in a precursor solution that is suitable for forming hydrogels ranges from about 10 to about 1×10⁸ cells/mL, from about 100 to about 1×10⁷ cells/mL, from about 1×10³ to about 1×10⁶ cells/mL, or from about 1×10⁴ to about 1×10⁵ cells/mL. In some embodiments, a single hydrogel comprises a population of identical cells and/or cell types. In some embodiments, a single hydrogel comprises a population of cells and/or cell types that are not identical. In some embodiments, a single hydrogel may comprise at least two different types of cells.

In some embodiments, a single hydrogel may comprise 3, 4, 5, 10, or more types of cells. To give but one example, in some embodiments, a single hydrogel may comprise only embryonic stem cells. In some embodiments, a single hydrogel may comprise both embryonic stem cells and hematopoietic stem cells.

### 3.2. Media

Any of a variety of cell culture media, including complex media and/or serum-free culture media, that are capable of supporting growth of the one or more cell types or cell lines may be used to grow and/or maintain cells. Typically, a cell culture medium contains a buffer, salts, energy source, amino acids (e.g., natural amino acids, non-natural amino acids, etc.), vitamins, and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites.

Cell culture media suitable for use in accordance with the present invention are commercially available from a variety of sources, e.g., ATCC (Manassas, Va.). In certain embodiments, one or more of the following media are used to grow cells: RPMI-1640 Medium, Dulbecco's Modified Eagle's Medium, Minimum Essential Medium Eagle, F-12K Medium, Iscove's Modified Dulbecco's Medium.

Those skilled in the art will recognise that the cells listed herein represent an exemplary, not comprehensive, list of cells that can be encapsulated within a precursor solution (and, therefore, eventually in a hydrogel) in accordance with the present invention.

### 4. Applications

The present invention aims to provide a filler that supports the natural healing of damaged tissue without inducing any specific tissue-formation. It is aimed to use the invention to fill a tissue cavity or cover a tissue defect to provide required filling space, partially or completely with minimal foreign body reaction.

Preferably, the composition of the invention is administered to a subject (e.g., a mammal) by injection or via spraying particles embodying the invention via aerosol or the like.

Uniquely, the composition of the present invention can be used to fill a defect, partially or completely, regardless of the shape and/or depth/size of the defect, can be added in a layer-by-layer fashion to build the required volume. The composition of the present invention adheres to the site without the need for physical containment, mixes with blood in situ and can be injected to deep tissue through a fine gauge needle. The tissue gap can be re-filled with the composition of the present invention at different time intervals as required.

The present invention provides an injectable filler. Upon the injection to the body the composition forms an adhesive hydrogel. Due to intrinsic properties of the invention, the composition of the present invention is well-tolerated in the body with minimal inflammatory response. The product is host tissue-conductive but not inductive as it only displays regenerative properties in the presence of an active bleeding or other fluids containing regenerative biological components.

The composition of the present invention can be injected through a fine gauge needle. The composition of the present invention adheres to the injection site and can be injected in a manner that creates a 3D structure; layer-by-layer inside the body through a minimally invasive manner.

Tissue types for which the present application is useful include dermal tissue and periodontal tissue.

Uses of the composition of the present invention include: a) an injectable dermal filler to fill skin cavity for temporary reduction of skin wrinkles (cosmetic use); b) an injectable dermal filler to temporarily lift the base of a scar and promote healing (therapeutic use); c) an injectable dermal filler to support dermal connective tissue formation in scar tissue after a surgical intervention and promote healing (therapeutic use); d) an injectable dermal filler to support dermal connective tissue formation in scar management of post burn injuries (cosmetic/therapeutic use); e) a ready to use dermal matrix to support vascular ingrowth in an acute dermal defect with bleeding and promote healing (therapeutic use); f) a ready to use dermal matrix to fill a surgically generated dermal cavity (therapeutic use); g) a ready to use dermal matrix to support skin grafting operation (cosmetic and therapeutic use); h) a carrier system to physically deliver bone graft substitutes (therapeutic use; see, e.g., Expert Rev Med Devices., 2006 January; 3 (1): 49-57); i) a carrier system to fill a prosthetic (e.g., cage) ex vivo and/or in vivo (therapeutic use); j) a filler with no tissue-inductive properties; k) a ready to use matrix to support and repair periodontal tissue after tooth extraction (therapeutic use); l) a ready to use matrix to support periodontal ligament tissue grafting (therapeutic use); and m) an injectable matrix to temporarily lift a periodontal ligament tissue (therapeutic use).

Advantages of preferred embodiments of the present invention include the following: a) cell-friendly; b) injectability; c) host tissue adhesivity; d) no tissue specific induction properties; and e) well-tolerated in the body (minimal immune response).

The hydrogel of the present invention preferably results in minimal foreign body reaction as a result of injection into an animal body.

The hydrogel of the present invention is preferably host-tissue conductive. This means that it displays regenerative properties in the presence of an active bleeding or other fluids containing regenerative biological component. The hydrogel of the present invention is preferably mixable with blood.

The composition of the present invention is preferably injectable. Preferably multiple injections to the same site are possible. The composition of the present invention preferably forms a hydrogel in situ after administration to a mammal by injection.

The hydrogel of the present invention preferably demonstrates good adhesion to host tissue. The adhesion properties allow an underlying tissue bed to gradually be built to support healing. The adhesion properties also allow formation of 3D structures in a minimally invasive manner.

The hydrogel of the present invention can preferably be used for layer-by-layer filling. This enables filling of 3D cavities with different heights.

The hydrogel of the present invention preferably degrades over a period of days, weeks or months, in situ, to leave healthy tissue.

The present invention has been developed as a safe and easy-to-use biomaterial as an injectable scaffold. The invention resides in a single uniform molecule comprised of a synthetic smart polymer (PNPHO) that is conjugated with Thymosin beta-4. The inventive composition is liquid at room temperature, enabling direct injection to the desired clinical location. The inventive composition forms an elastic gel on exposure to body temperature, mixes with blood and stabilises the clot at the site. In addition, the resorption rate of the inventive composition, based on in vitro and in vivo studies, is thought that the inventive composition resorbs to the body in less than three months. In addition, sheep osteotomy model and osteoblast and pre-osteoblast cell studies confirm that the device supports vascular ingrowth and bone formation.

It was hypothesised that the inventive composition may be injected into a socket base post-tooth extraction to mix with the blood, stabilise the clot and provide a uniform scaffold for vascular ingrowth and bone regeneration. It was aimed to enhance wound healing at the site and preserve alveolar bone post tooth extraction by direct administration of the inventive composition to extraction socket. To investigate this hypothesis, Applicant carried out a clinical trial that involved ten participants with scheduled tooth extraction. Soft tissue closure (would healing), appearance of the extraction site, well-being of participants as well as the quality of the underlying bone were studied in this clinical investigation.

Applications of the inventive composition and the presence of supporting data for these potential uses of the technology are summarised in Table 1.

TABLE 1
Possible applications of the inventive composition and
current supporting preclinical results

| # | Potential applications | Supporting results |
|---|---|---|
| 1 | An injectable dermal | Animal results, mice subcutaneous |
| | filler to fill skin | model (Study 001); |
| | cavity for temporary | injection of PNPHO-co- |
| | reduction of a skin | TB4 with no surgical |
| | wrinkle | intervention (no bleeding) resulted |
| | | in the formation of an |
| | | adhesive inert structure under |
| | | skin for at least 1 month. |
| | | No regeneration noted after |
| | | this time point and no |
| | | inflammatory response, or |
| | | foreign body reaction noted |
| 2 | An injectable dermal | Animal results, mice subcutaneous |
| | filler to temporarily | injection (Study 001) |
| | lift the base of a scar | showed the injectability of the |
| | in skin | technology under skin and |
| | | its full resorption after 1 month |
| | | with no inflammatory |
| | | response or foreign body |
| | | reaction noted |
| 3 | An injectable dermal | Animal results, mice dermal |
| | filler to support | tissue use (Study 002) with |
| | dermal connective | a dermal defect showed the |
| | tissue formation in a | formation of connective |
| | scar tissue after a | dermal tissue at the site of |
| | surgical intervention | administration. There was |
| | | minimal inflammation observed |
| 4 | A ready to use dermal | Animal results, mice dermal |
| | matrix to support | tissue use (Study 002) with |
| | vascular ingrowth | a dermal defect showed the |
| | in an acute dermal | formation of blood vessels |
| | defect (with bleeding) | after 2 weeks and 4 weeks |
| | | post-administration of |
| | | PNPHO-co-TB4 |
| 5 | A ready to use dermal | Animal results, mice dermal |
| | matrix to fill a | tissue use (Study 002) |
| | surgically generated | during the operation showed |
| | dermal cavity | that PNPHO-co-TB4 can |
| | | adhere to the site and skin can |
| | | be sutured over the defect |
| | | site |
| 6 | A ready to use dermal | Animal results, mice dermal |
| | matrix to support | tissue use (Study 002), |
| | skin grafting operation | PNPHO-co-TB4 support 100% |
| | | of skin grating operation |
| | | compared with 82% success |
| | | rate in the treatment group |
| | | with the gold standard |
| 7 | A carrier system to | Animal results, sheep osteotomy |
| | physically deliver | model (Study 003), |
| | bone graft substitutes | PNPHO-co-TB4 was used to |
| | | deliver demineralised bone |
| | | matrix to an osteotomy site |
| 8 | A carrier system to | Benchtop testing, filling of |
| | fill a prosthetic (e.g., | a spinal cage with PNPHO- |
| | cage) ex vivo and in vivo | co-TB4 combination with |
| | | demineralised bone matrix |
| 9 | A filler with no | Animal results, intramuscular |
| | tissue-inductive | injection of PNPHO-co- |
| | properties | TB4 with and without BMP-2 |
| | | and showing no ectopic |
| | | muscle ingrowth in PNPHO- |
| | | co-TB4/−BMP2 and ectopic |
| | | bone formation in PNPHO- |
| | | co-TB4/+BMP2 group |
| 10 | A ready to use matrix | Clinical results from PET-A, |
| | to support repair | 10 patients and healthy soft |
| | periodontal tissue after | tissue regeneration and |
| | tooth extraction | wound closure after tooth |
| | | extraction |
| 11 | A ready to use | Clinical results from PET-A, |
| | matrix to support | 10 patients and healthy soft |
| | periodontal ligament | tissue regeneration and |
| | tissue grafting and | wound closure after tooth |
| | matrix to temporarily | extraction |
| | lift periodontal | |
| | ligament tissue | |
| 12 | A filler to use as a | Clinical results from PET-B, |
| | carrier system for | mixture of PNPHO-co-TB4 |
| | bone graft substitutes | with BioOss (xenograft) and |
| | | Rabbit femoral defect |
| | | model, mixture of PNPHO- |
| | | co-TB4 with demineralised |
| | | bone matrix (DBM) |
| 13 | Aerosol formation | Preclinical proof of concept |
| | with diluted hydrogel | bench-top testing |

### 5. Kits

Disclosed herein are a variety of kits comprising one or more of the preferred hydrogels for use in the present invention. For example, the invention provides a kit comprising a hydrogel and instructions for use in repairing or regenerating bone defects. A kit may comprise multiple different hydrogels.

A kit may optionally comprise polymers, cells, NSPP(s), biologically-active compounds, water, and the like. A kit may comprise any of a number of additional components or reagents in any combination. All of the various combinations are not set forth explicitly, but each combination is included in the scope of the invention. Some exemplary kits that are provided in accordance with the present invention are described in the following paragraphs.

According to certain embodiments of the invention, a kit may include, for example:
i. a solution comprising a polymer, a solution comprising NSPP; and
ii. instructions for forming a hydrogel from the solution.

According to another embodiment, a kit may include, for example:
i. a composition comprising a polymer and NSPP; and
ii. instructions for forming a hydrogel from the composition.

According to another embodiment, a kit may include, for example:
i. a composition comprising a polymer and NSPP, one or both being in solid form; optionally a solvent such as water or the like; and
ii. instructions for forming a hydrogel from the composition.

Kits may further include other materials desirable from a commercial and user standpoint, including other buffers, diluents, filters, needles, and syringes. Exemplary kits can be in the form of an aerosol, or with components combinable to form an aerosol or similar means of applying the composition of the invention.

Kits typically include instructions for use of the preferred hydrogels for use in the present invention. Instructions may, for example, comprise protocols and/or describe conditions for production of hydrogels, administration of hydrogels to a subject in need thereof, production of hydrogel assemblies, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as Styrofoam, etc., may be enclosed.

The kit or “article of manufacture” may comprise a container and a label or package insert on or associated with the container. Suitable containers include, for example, bottles, vials, syringes, blister packs, etc. The containers may be formed from a variety of materials such as glass or plastic. The container holds the hydrogel or composition which is effective for treating the condition and may have a sterile access port (for example the container may be an intravenous solution bag or a vial having a stopper pierceable by a hypodermic injection needle). The label or package insert indicates that the hydrogel or composition is used for treating the condition of choice. In one embodiment, the label or package insert includes instructions for use and indicates that the therapeutic composition can be used to repair or regenerate tissue.

### Preferred Embodiments of the Invention

The preferred polymer used in the invention is PNPHO. The concentration of PNPHO in the composition of the invention is preferably from about 100 to about 300 mg/mL of the composition. When embodied in aerosol form, the concentration is about 5 mg/mL to about 70 mg/mL of the composition.

The preferred NSPP used in the invention is Thymosin beta-4. Alternatively a functional homolog of Thymosin beta-4 may be used.

Preferably the PNPHO is conjugated with Thymosin beta-4 (or a functional homolog thereof), where both the protein/peptide segment and PNPHO have defined roles.

The Thymosin beta-4 (or functional homolog thereof):
i. serves as the source of bioactive signalling for tissue regeneration; and
ii. promotes the formation of blood vessels within and around the hydrogel filler.

The PNPHO polymer is chemically-bonded with protein/peptide to:
i. adjust the physicochemical properties of this biopolymer for tissue applications;
ii. impart rapid thermosetting to the hydrogel filler to confine it locally; and
iii. impart bioresorption properties to the injectable hydrogels.

The combination of these two main segments results in the formation of the new class of smart tissue fillers with a range of favourable properties for tissue regeneration and repair. An advantage of the PNPHO polymer is that all its components are approved by the United States FDA for use in biomedical applications.

The PNPHO polymer comprises a thermally responsive fraction (N-isopropyl acrylamide) to induce the hydrogel formation at body temperature, along with lactide, ethylene glycol and N-acryloxysuccinimide segments to impart respectively, a high mechanical strength, water solubility, and amine group reactivity to the product. The molecular structure of the PNPHO polymer and the role of each segment are schematically shown in the schemes drawn below in the Examples.

Preferably, equimolar amounts of PNPHO and Thymosin beta-4 are used, although one of ordinary skill in the art will appreciate that the molar ratio may be varied according to each scenario encountered in practice.

### EXAMPLES

### Materials

Chemicals were purchased from Sigma-Aldrich unless otherwise stated. Stannous 2-ethylhexanoate (Sn(Oct)₂), N-isopropylacrylamide (NIPAAm), 2-hydroxyethyl methacrylate (HEMA), 4,4′-azobis(4-cyanovaleric acid) (ACVA) and N-acryloxysuccinimide (NAS) were used as received. Oligo (ethylene glycol) monomethyl ether methacrylate (OEGMA, Mₙ=475) was purified by passing its solution in dichloromethane (with 1:1 volume ratio) through a neutral alumina column to remove the inhibitor prior to use. D, L-lactide (LA) monomer was dried under vacuum at 40° C. for 24 hours prior to use. Azobisisobutyronitrile (AIBN) was kindly gifted by School of Chemistry in the University of Sydney, Australia.

### Synthesis of HEMA-Poly(Lactide) (HEMA-PLA) Macromonomer

HEMA-PLA macromonomer was synthesised by ring-opening polymerisation of LA with the hydroxyl group of HEMA as the initiator and Sn(Oct)₂ as the catalyst (see, Scheme 1).

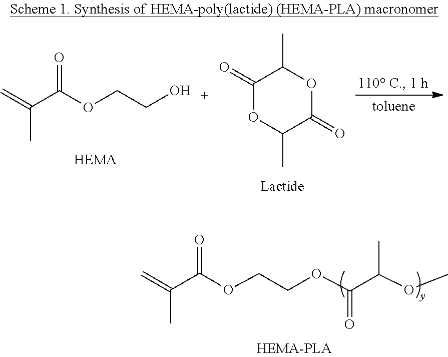

LA and HEMA were mixed in a three-neck flask at 110° C. under a nitrogen atmosphere for 15 minutes. Subsequently, a mixture of 1 mol % of Sn(Oct)₂ (with respect to the HEMA feed) in 1 ml of anhydrous toluene was added to the LA/HEMA solution. The resulting mixture was stirred at 300 rpm and 110° C. for 1 hour under a nitrogen atmosphere. After the reaction, the mixture was dissolved in tetrahydrofuran and precipitated in cold distilled water at 1° C. The formed precipitate was separated by centrifugation at 3000 rpm for 5 minutes.

The centrifugation cycle was repeated three times to remove all unreacted monomers and by-products (mainly salts). The precipitate was then dissolved in ethyl acetate. The suspended solid particles were removed from the solution with centrifugation at 6000 rpm for 5 minutes and the supernatant was dried with MgSO₄ for 12 hours. The dried supernatant was filtered to remove the MgSO₄ particles. The polymeric solution was then dried at 60° C. under reduced pressure and the residue of solvent was further removed under vacuum, at 40° C. for 24 hours. The resultant viscous oil was stored in a fridge for further use.

The feed ratio of HEMA:LA was varied from 1:1.5 and 1:2.5 to obtain PLA/HEMA macromonomers with different lactate lengths. Two PLA/HEMA macromonomers with lactate lengths of 3 and 6 were synthesised by using 1:1.5 and 1:2.5 mol ratio of HEMA to LA monomers, respectively.

The synthesis of PLA/HEMA macromonomer was confirmed, using ¹H NMR spectra with evidence of proton peaks from both HEMA and LA. The molar ratio of LA to HEMA in the PLA/HEMA macromonomer was calculated from 1H NMR spectra using the peaks at 5.2 ppm for methine in lactate, and total integrations at 5.7 ppm and 6.0 ppm peaks for HEMA.

### Synthesis of Poly(NIPAAm-co-NAS-co-(HEMA-PLA)-co-OEGMA) (PNPHO)

PNPHO was synthesised using either method (1) or (2) as described below (see, Scheme 2).

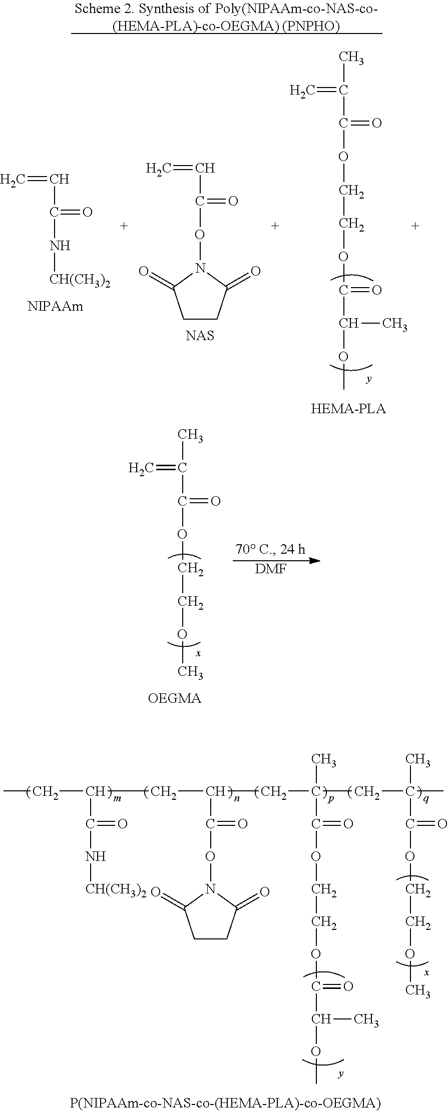

### Method 1

PNPHO was synthesised by free radical polymerisation using AIBN as the initiator. A Schlenk flask with a magnetic stir bar and a rubber septum was charged with NIPAAm (12 mmol), NAS (1.0 mmol), HEMA-PLA (0.57 mmol), OEGMA (0.56 mmol), AIBN (0.07 mmol) and anhydrous N,N′-dimethylformamide (DMF). The flask was deoxygenated by three freeze-pump-thaw cycles, and then sealed followed by immersing the flask into an oil bath preheated at 70° C. to start the polymerisation. After 24 hours, the reaction mixture was cooled to room temperature, precipitated in diethyl ether, filtered, and then dried under vacuum. The polymer was purified twice by redissolving/reprecipitating with THF/ethyl ether and finally dried under vacuum for 2 days.

### Method 2

PNPHO was synthesised by free radical polymerisation, using ACVA as the initiator. The composition of the copolymer was changed by varying the lactate length (3 and 6 in HEMA-PLA) and the molar ratios of HEMA-PLA (6, 8, and 11 mol %) and OEGMA (3, 5, and 8 mol %). Known amounts of NIPAAm, NAS, HEMA-PLA, OEGMA, ACVA (7.0×10⁻⁵ mol) were dissolved in 13 ml anhydrous N,N′-dimethylformamide in a round bottom, one neck flask. The system was then deoxygenated by 15 minutes of nitrogen purging. The results also showed that it is feasible to deoxygenate the monomer solution by purging nitrogen gas for 10 minutes in the solution under vacuum. This technique provides a more efficient method to remove oxygen from solution in large scales.

The reactor was then sealed and immersed in an oil bath at 70° C. for 24 hours. The resultant polymeric solution was then cooled at room temperature for 1 hour and precipitated in 250 mL diethyl ether. The precipitate was then collected by filtering the suspension and dried under vacuum for 6 hours. The dried powder was dissolved in tetrahydrofuran and precipitated in diethyl ether to further remove residues of macromonomers. The final powder was dried under vacuum for at least 48 hours.

TABLE 2
PNPHO polymers (in the form OEGMA:PLA/HEMA(LA
length):NAS:NIPAAm)

| Monomer Feed | Final Composition | Gelation Time (min) |
|---|---|---|
| 5:8(5):3.5:83 | 4.8:7.9(5):7:80 | 2.5 ± 0.6 |
| 5:8(5):7:80 | 4.6:7.8(5):13.6:74 | 3.1 ± 0.9 |
| 8:6(5):3.5:82 | 8:5.4(5):7:80 | 5.1 ± 0.6 |
| 8:6(5):7:80 | 8:5.1(5):14:72 | 5.4 ± 0.1 |
| 3:8(5):3.5:72 | 4.8:7.9(5):7:80 | 2.0 ± 0.2 |
| 3:8(5):7:80 | 4.6:7.8(5):13.6:74 | 2.2 ± 0.9 |

### PNPHO Compositions

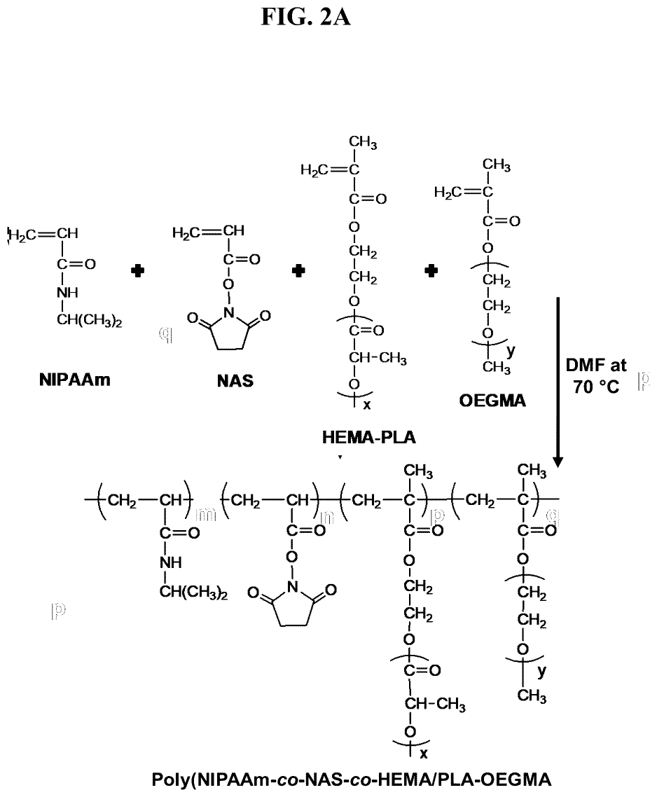

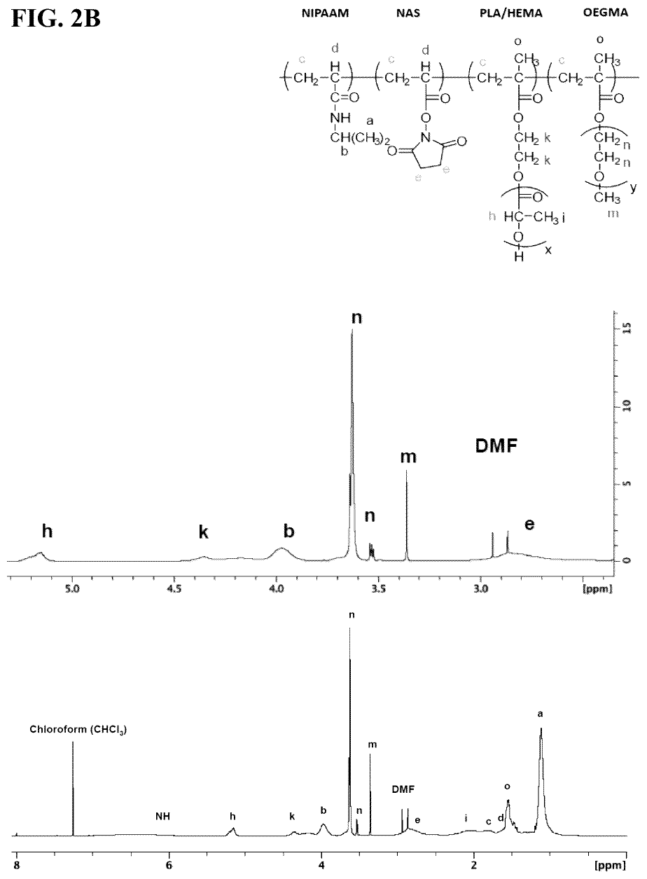

PNPHO was synthesised in accordance with the scheme shown in FIG. 2(a). The synthesis of PNPHO copolymers was confirmed with 1H NMR spectra with evidence of proton peaks for each monomer, as shown in FIG. 2(b). Characteristic proton peaks were detected for NIPAAm (a and b), NAS (e), HEMA-PLA (f, h, k), and OEGMA (m and n). In respect of the NAS proton resonances (e), these have been noted to overlap with resonances about 2.9-3.0 ppm for residual trace DMF solvent, making original calculations erroneous based on the same data set (integral/area beneath the respective peaks as basis for relative mol % of the respective monomers within the overall PNPHO polymer). Subsequent correction confirms that the third monomer (NAS) is present in an amount of greater than about 7 mol %. The final composition of copolymer was calculated based on the integration of these peaks from each monomer as for NIPAAm (a), NAS(e/2-f), HEMA-PLA (h), and OEGMA (n/2). In this study copolymer is denoted as PNPHO and the subscript is added that corresponds to HEMA-PLA (lactate length) to OEGMA molar ratios. For example PNPHO 5:8(5):7:81 stands for the copolymer synthesised with 8 mol % HEMA-PLA with lactate length of 5, and 5 mol % OEGMA. Various copolymers were produced. These are shown in Table 2. Table 2 also provides data on their gelation time and temperature.

### Solubility of PNPHO in PBS

The monomer ratios of PNPHO were modified to acquire a range of compositions that were dissolved in aqueous media, such as PBS for the development of injectable formulations. NIPAAm-based copolymers are soluble in aqueous solutions below their LCST due to the formation of hydrogen bonds between the copolymer polar groups and water molecules. In this study the effects of lactate length, HEMA-PLA and OEGMA contents on the solubility of PNPHO were studied by measuring the saturation concentration of different compositions of PNPHO in PBS.

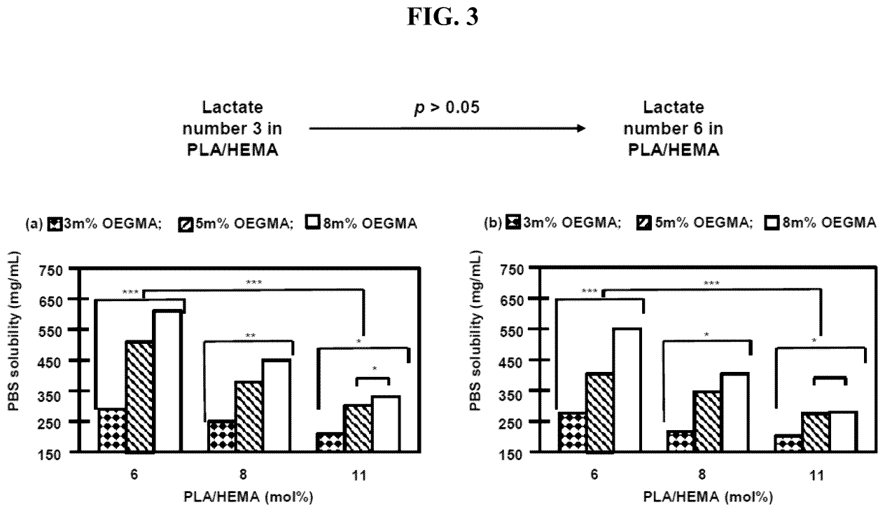

The results in FIG. 3 demonstrate that increasing lactate length within the range of 3 to 6 in the HEMA-PLA backbone had no significant impact on the solubility of PNPHO in PBS (p>0.05). Hydrophobic properties of a side chain in the backbone of PNPHO therefore had minimal impact on overall solubility of PNPHO in aqueous media, within the range examined. Therefore, by changing the lactate length, other characteristics of PNPHO, such as gelling behaviour and mechanical properties, can be tuned without affecting the solubility of PNPHO in an aqueous media.

The solubility of PNPHO in PBS can be tuned by changing both hydrophobic and hydrophilic contents. The PLA segment is the main hydrophobic backbone, while both NAS and HEMA monomers exhibit relatively limited hydrophilic properties. OEGMA was therefore included in the synthesis of PNPHO to promote the hydrophilic properties of the copolymer.

Increasing HEMA-PLA (i.e., the hydrophobic content) in copolymers from 6 to 8 and 11 mol % decreased the solubility of PNPHO in PBS by 30% and 50%, respectively. This solubility reduction was also due to decreasing the concentration of the relatively hydrophilic segment NIPAAm in the copolymer (p<0.05). Therefore, decreasing NIPAAm content in PNPHO substantially affected the hydration of the copolymer.

The solubility of PNPHO in water was increased dramatically, when using more than 3 mol % (e.g., 1.5 mol %) OEGMA as a hydrophilic segment. Results showed that copolymers with OEGMA contents of less than 3 mol % were not soluble in aqueous media. The results in FIG. 3 show that the solubility of PNPHO copolymers with 6 mol % PLA-HEMA was significantly increased 2- and 3-fold when elevating the OEGMA concentration from 3 mol % to 5 and 8 mol %, respectively. However, in copolymers that contained a higher molar ratio of hydrophobic segment HEMA-PLA (i.e., 8 mol % and 11 mol %), OEGMA concentration had little effect on the solubility of PNPHO. This behaviour was attributed to formation of copolymers with longer chains and higher Mw, which impeded the hydration and solubility of the copolymer in aqueous solution.

The effect of concentration of water soluble PNPHO copolymers on the injectability of their solutions through an 18 G needle was assessed. It was found that 150 mg/mL PNPHO solution in PBS was injectable through 18 G needle and this concentration of copolymer was used for further analysis. Higher concentrations of polymers can be used for other biomedical applications such as scaffold fabrication for in vitro tissue growth.

### Thymosin Beta-4

Since the succinimide linker exhibits high reactivity and optimised accessibility towards compounds containing amino groups, it is reasonable to postulate that the polymer can be applied to TB4 with amino groups for the fabrication of flowable hydrogels. To confirm this assumption, the feasibility of a reaction between the polymer and TB4 was examined. A TB4 solution was formed in phosphate buffered saline. 1000 μL of the TB4 solution was thoroughly mixed with 140 mg of polymer at 4° C. for at least 24 hours, the mixture was then allowed to gel at 37° C.

### Gelling Behaviour (Time and Temperature)

The results in Table 2 showed that the gelling time of the hydrogels fabricated with PNPHO composition were within the range of 2.5 to 5 minutes. It is important to note that this measurement was carried out in simulated physiological condition (37° C. incubator) and the gelation rate of the hydrogel in contact with body fluid and/or body surface is significantly faster at this temperature. This gelation time is favourable for clinical applications as it allows the administration of the product and at the same time prevent the dislocation of the administered product from the site. In addition, the gelation temperatures of these formulations of PNPHO are below 37° C. which allows their clinical use. The high gelation temperatures of the formulations are of great convenient for clinicians to deliver the solutions in vivo.

### Conjugation Efficiency of PNPHO

In order to test the conjugation efficiency of PNPHO, TB4 synthetic peptide was used as a model system. The results indicated that TB4 was effectively conjugated with different PNPHO formulations. This result showed the high efficacy of the PNPHO to conjugate synthetic or natural peptides or proteins. The two components (TB4 and PNPHO) interact in molecular level via the succinimide ester groups of PNPHO and amino groups of TB4. To assess this, the lower critical solution temperature (LCST) of PNPHO and PNPHO-co-TB4 are compared.

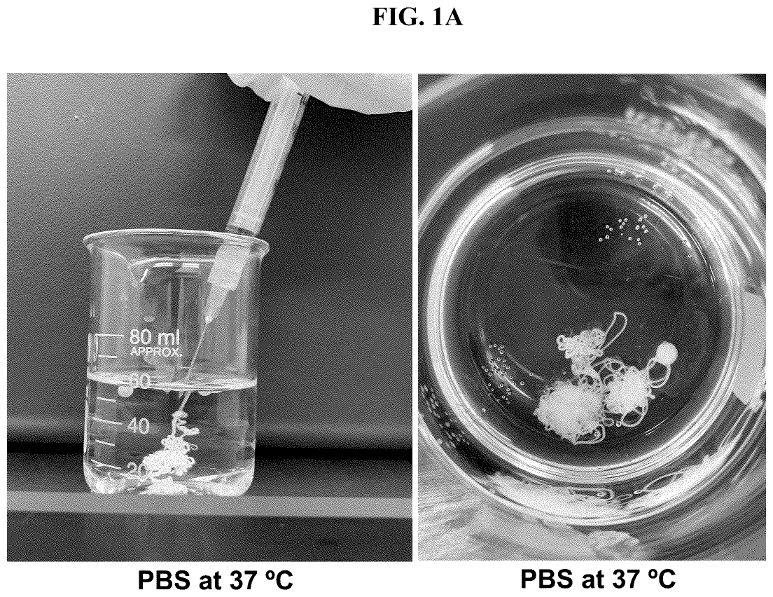

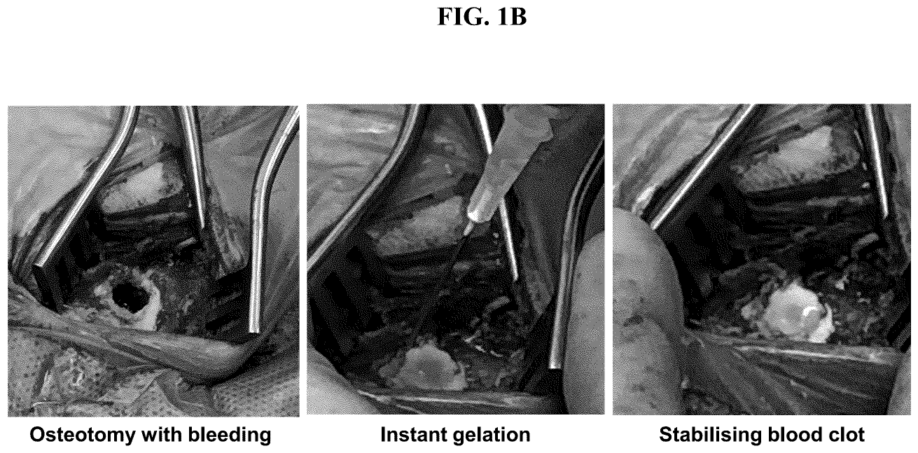

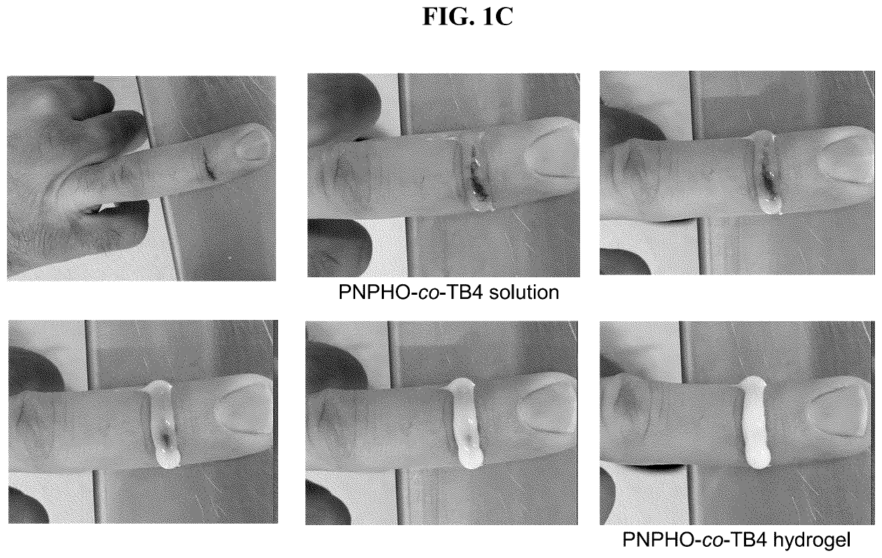

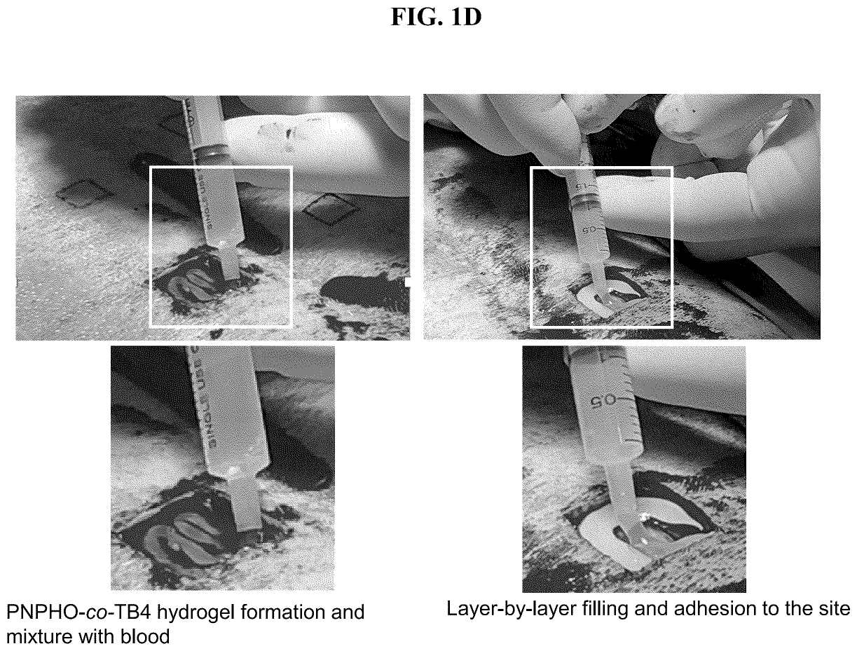

The LCST of the PNPHO polymer and the conjugated system (PNPHO-co-TB4) is driven by the chemical composition of PNPHO, TB4 and the ratio between hydrophilic and hydrophobic groups in the molecular structure. PNPHO and PNPHO-co-TB4 exhibit reverse solubility upon heating. This thermoresponsive behaviour arises from the ability of NIPAAm groups and the associated side chains, e.g., NAS, PLA/HEMA, OEGMA and TB4) to undergo a change from a dissolved coil to a collapsed globule (i.e., transition from hydrophilic to hydrophobic state) when the temperature is raised above the LCST (FIG. 1). ¹H-NMR provides an accurate profile of the LCST due to monitoring the transition of polymer during the temperature change of NMR data acquisition. ¹H-NMR spectra are recorded at a range of temperatures from 10° C. to 30° C. at 1° C. intervals. As the temperature increases and upon coil-to-globe transition of the conjugate system (reverse solubility), the NMR signal drops for various peaks. The plot of NMR peak areas versus temperature identifies the LCST.

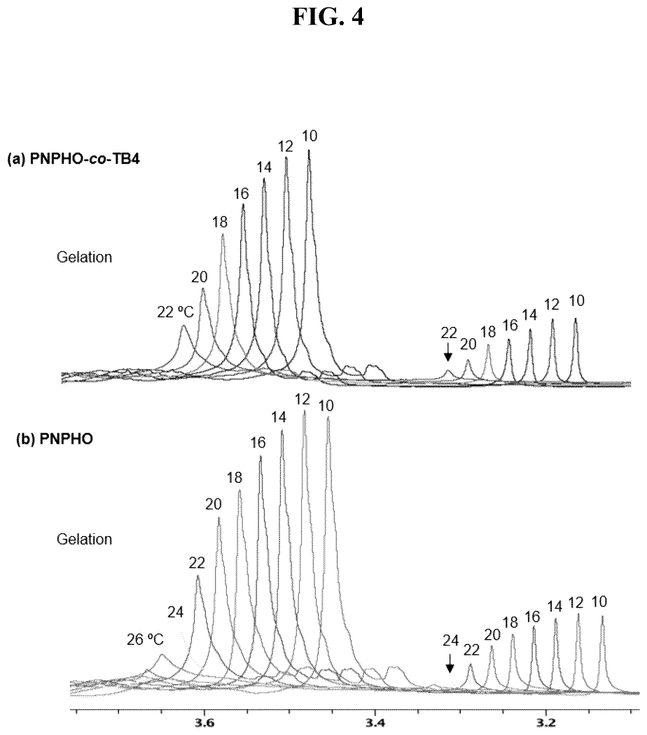

Based on the ¹H-NMR spectra of PNPHO polymer and PNPHO-co-TB4, it is found that LCST of the conjugated system is 2 to 4° C. lower than that of for PNPHO polymer (FIG. 4). To quantify the gelation temperature D₂O/m (characteristic peak in relation to OEGMA) was used. The shift in the gelation temperature of the conjugated system in comparison with PNPHO confirmed the presence of chemical interaction between the two components. In addition, decrease of gelation temperature increases the kinetic of gelation in vivo and thus facilitates the adhesion of the product at the administration site.

### Bioresorbable Behaviour of Protein-PNPHO Hydrogels

The clinical study of PNPHO-co-TB4 showed that the product was fully resorbed to the body within 3 months. Histological evaluation of the implantation site showed that there is no trace of the product three to four months post implantation.

### Feasibility Study

TABLE 3
Product configurations applied in feasibility study

| Product Configuration(s) | Other Variable(s) |
|---|---|
| PNPHO-co-TB4 (140 mg/mL of PNPHO | Skin Graft indication |
| 5:8(5):7:81 and 30 mg/mL of Thymosin | Dental indication |
| beta-4 (TB4)) | |

Samples from the batch were incubated on 90 mm trypcase soy agar plates at 37° C. Absence of bacterial growth was verified prior to batch release being performed.

The sample size(s) that were used in execution of this protocol is/are shown in Table 4.

TABLE 4
Sample sizes used in test protocol

| | | Number of tests per |
|---|---|---|
| Test(s) being | Sample size i.e., # | test article (D = Day |
| Performed | of test articles | W = Week) |
| Skin graft | Total = 20 | 1 per each |
| survival | n = 20 up-to 2 weeks | time point |
| rate | post-operation (2 W-PO) | Time points = 1 D, |
| | n = 14 up-to 4 weeks | 3 D, 1 W, |
| | post-operation (4 W-PO) | 2 W, 3 W, |
| | n = 8 8 weeks post- | 4 W, 6 W, 8 W |
| | operation (8 W-PO) | |
| | Note: 6 animals | |
| | will be sacrificed | |
| | 2 weeks and 4 weeks post- | |
| | operation; the remaining 8 | |
| | animals will be sacrificed 8 | |
| | weeks PO | |
| | 2 extra animals (replacement | |
| | subjects) for 8 weeks post- | |
| | operation time-point due to | |
| | high risk of natural death for | |
| | animal in 8 weeks | |
| Blood vessel | N = 8 animals All animals in | 1 per each |
| ingrowth | 8-weeks post-operation time | time point |
| | point will be scanned for live | Time points = |
| | animal fluorescent radiant | 2 W, |
| | efficiency testing at different | 4 W, 6 W |
| | time points; e.g., 2 weeks post, | |
| | 6 weeks post and 8 weeks post | |
| Foreign body | n = 6 for each time point If not | 2 per each |
| reaction, cell | used, two extra animals | time point |
| infiltration, | (replacement subjects) will be | Time |
| extracellular | sacrificed in accordance with | points = 2 W, |
| matrix | pre-approved animal ethics | 4 W, 6 W |
| formation and | | |
| angiogenesis | | |

The sample size(s) selected for all tests to be performed have been selected with consideration of statistical validity. Significant number of animals/test articles and control groups are selected, e.g., 6 repetitions per group per time point which is well above 3 repetitions, normally used in preclinical, proof of concept studies).

TABLE 5
Test protocols employed

| Test(s) | | Number of | |
|---|---|---|---|
| being | Sample size i.e. | tests per | |
| performed | # of test articles | test article | Sample size justification |
| Skin graft | N (2 WK-PO) = 20 | 1 | Minimum 3 required for |
| survival rate | N (4 WK-PO) = 14 | 1 | pre-clinical animal study |
| | N (6 WK-PO) = 8 | 1 | |
| Blood vessel | n = 10 | 1 | Minimum 3 required for |
| ingrowth | | | preclinical animal study |

PNPHO-co-TB4 test articles were manufactured by Applicant in accordance with documented procedures and processes. All equipment, tools and materials used during product manufacture were approved within Applicant's QMS. PNPHO-co-TB4 devices were supplied to the testing facility in ready-to-use syringes. Each test article was single use.

The following equipment/tools/materials were required in the execution of this test protocol. Histochemical analyses, staining and visualisation were carried out in accordance with industry standards

TABLE 6
Equipment/tools/materials required in the execution of test protocol

| | | Quantity |
|---|---|---|
| Equipment Name | Equipment Number | Used |
| IVIS Lumina XR live imager | N/A (external equipment) | 1 |
| | | Quantity |
| Material Name | Material Number | Used |
| IVIS Lumina XR live imager | AngioSense750 EX in vivo | 20 |
| Probe> | blood pool fluorescent | |

### Results

PNPHO-co-TB4 solution transitions from liquid at room temperature (20 to 25° C.) to gel at body temperature (37° C.) and it retains its structural stability after injection in a simulated physiological condition (FIG. 1(a)). A live sheep osteotomy model was used to examine injectability and adhesion of PNPHO-co-TB4 in the presence of active bleeding. The results in FIG. 1(b) show that the product can be injected to the defect site and it instantly forms a hydrogel which fills the cavity, mixes with the blood and stabilises the clot. The hydrogel formation was achieved despite active bleeding at the site and consequent dilution of the hydrogel with blood. This is a significant attribute of the inventive hydrogel system as it is useful for clinical applications of injectable systems and to prevent device malfunctions. Better heat transfer due to the presence of body fluid in in vivo injection promotes the kinetics of hydrogel formation. For instance, FIG. 1(c) shows that the hydrogel formation on the skin is slower. This characteristic behavior of the invention can be used to form 3D structures via layer-by-layer administration of the product (FIG. 1(d)) on exposed wounds, which normally have lower temperature compered to core/deep tissue. The invention is adhesive and mixes with the blood and host cells to support healing.

The ability of PNPHO-co-TB4 to support tissue regeneration is based on the physical scaffold formed upon administration. This matrix is suggested to support cell infiltration and vascular ingrowth throughout its structure. To test this hypothesis, a mice animal model is used to investigate the in vivo potentials of PNPHO-co-TB4 to integrate with the host environment and support vascular ingrowth. In addition, PNPHO-co-TB4 was directly compared to Integra Regenerative Dermal Matrix (Integra) as the positive control.

### Animal Model and Surgery

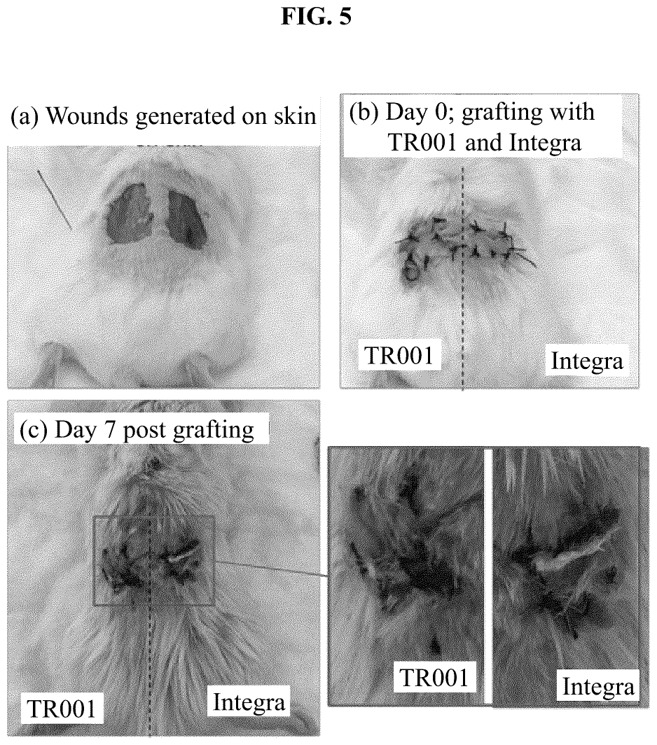

Full thickness mice animal model (n=20) is used; in each animal, two (2) full-thickness dermal defects were surgically generated; see, FIG. 5(a). Wounds were treated with PNPHO-co-TB4 or Integra (positive control) and skin grafts sutured in place as shown in FIG. 5(b). Routine dressing was then applied to treatment site, specifically Atrauman and IV 3000. Blood vessel formation and ingrowth at the grafted sites were determined by fluorescent radiant efficiency using an IVIS Lumina XR live imager. Defects were checked regularly up to 8 weeks post treatment for graft survival. In addition, animals were sacrificed at different timepoints; biopsies were collected to quantify host tissue integration, vascular ingrowth and inflammatory response.

### Skin Graft Survival

The survival of the grafted skins at different time points throughout the study was investigated. A successful skin graft remains intact with the host dermal site whereas a failed site folds and overtime detaches; see, FIG. 5(c). Results from this investigation showed that within 1-week post operation, 4 skin grafts treated with Integra failed, indicating the survival rate of 80%. This finding is in agreement with previous studies, reporting the survival rate of approximately 85% for Integra (“Design of an artificial skin. I. Basic design principles,” n.d.; Vyas & Vasconez, 2014). All grafted sites treated with PNPHO-co-TB4 survived throughout the study; 100% survival rate of skin grafts treated with PNPHO-co-TB4. The efficacy of PNPHO-co-TB4 to support full-thickness skin graft suggest that PNPHO-co-TB4 is well-tolerated with minimal inflammatory response. Successful skin graft survival suggests PNPHO-co-TB4 acts as regenerative scaffold for neo-vascular ingrowth, host tissue infiltration and extracellular matrix formation. To investigate these characteristics of PNPHO-co-TB4, live fluorescent imaging of animals at different time points and histological evaluations of the treated sites were conducted.

### Inflammatory Response to the Inventive Composition

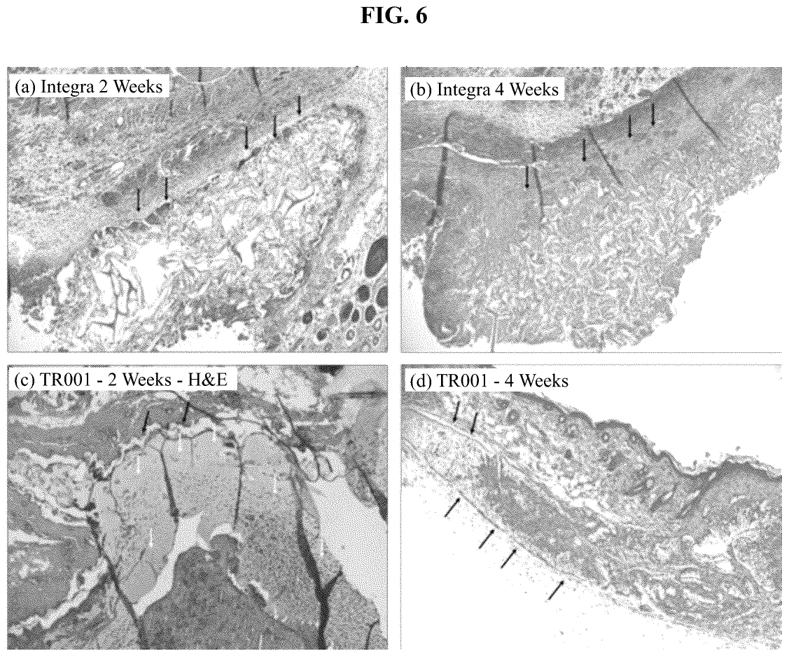

Skin biopsies were collected two (2) and four (4) weeks post grafting to histochemically assess the biological behaviour of PNPHO-co-TB4. H&E staining of the sites treated of Integra, shows mild inflammatory responses to the Integra constructs at both 2 and 4 weeks; FIG. 6(a) and FIG. 6(b), respectively, shown by the multi-layered fibrous tissue visible around Integra sites. Previous studies, have shown similar inflammatory responses to constructions and the formation of fibrous tissue around Integra (Hori, Osada, Isago, & Sakurai, 2017; Y. Wang, et al., 2015).

In contrast, H&E staining of the skin biopsies of PNPHO-co-TB4 treated sites in FIG. 6(c) and FIG. 6(d) show a very narrow layer of fibrous tissue was formed around PNPHO-co-TB4, two weeks post grafting. White arrows in FIG. 6(c) showed the structure of PNPHO-co-TB4 gel and black arrows identify minimal fibrous tissue formation around PNPHO-co-TB4 hydrogel. Recognising that increased inflammation is correlated with impaired wound remodelling, the reduced fibrous tissue formed around PNPHO-co-TB4 suggests the material is conducive to effective soft tissue repair.

### Neo-Vascularisation

Blood vessel formation and ingrowth at the grafted sites were determined by fluorescent radiant efficiency using an IVIS Lumina XR live imager. Two (2) and four (4) weeks post grafting operation, angiogenic responses were determined with an AngioSense750 EX in vivo blood pool fluorescent imaging probe. This near-infrared fluorescent macromolecular probe persists in the vasculature and enables imaging of blood vessels and angiogenesis. At each time point after the surgery, each mouse was injected with 2 nmol AngioSense750EX in 100 μL PBS. After 24 h, each mouse was scanned for fluorescent radiant efficiency (n=8).

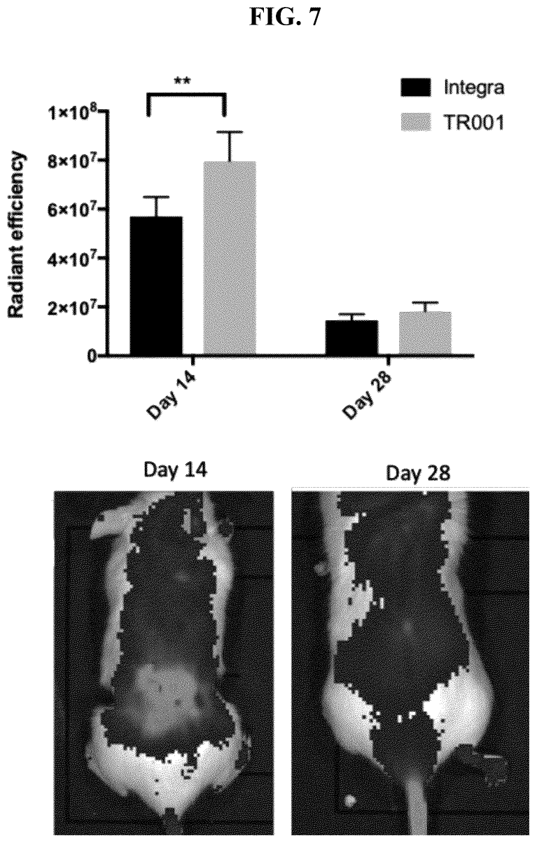

The radiant efficiency was used to indicate the density of new blood vessels in wound area. Results in FIG. 7 showed that two weeks post operation, the fluorescent radiant efficiency on PNPHO-co-TB4 treated site was significantly higher (p 0.01) that of Integra treated site. In contrast, angiogenic signals were very low for both treatment groups four (4) weeks post operation, indicating that the blood vessel formation was controlled and the healing of the site was complete. Similar findings are reported in the previous studies, indicating that the treatment sites after four weeks display no angiogenic signals (Yiwei Wang, et al., 2015).

Vascular Ingrowth and Cellular Infiltration within the Inventive Composition

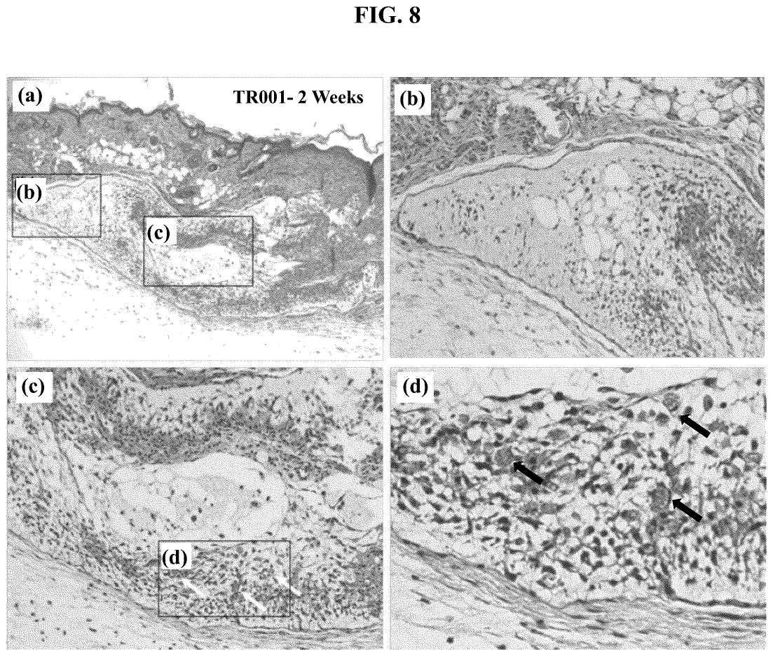

Histological evaluation of the PNPHO-co-TB4 treated sites four (4) weeks post skin grafting confirmed the potential of PNPHO-co-TB4 to support vascular ingrowth and host cell infiltration. The H&E staining of the skin biopsies are shown in FIG. 8. As previously outlined, a thin layer of fibrous tissue was formed around PNPHO-co-TB4 structure, indicating that the injectable hydrogel is well-tolerated in the body; FIG. 8(a) and FIG. 8(b). More importantly, this thin layer of fibrous tissue can be used as indication of PNPHO-co-TB4 boundaries in dermis.

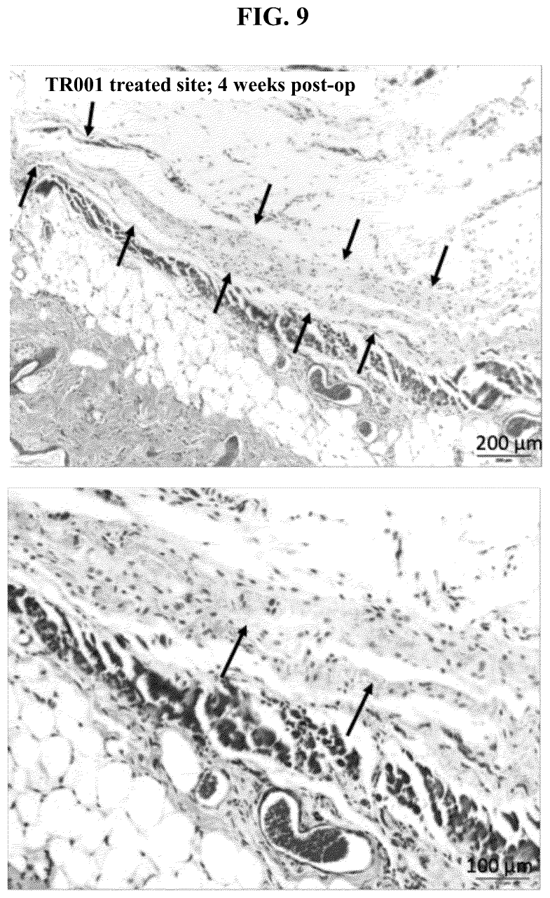

Results in FIG. 8(c) and FIG. 8(d) show the formation of blood vessels within the structure of PNPHO-co-TB4. In addition, the staining of the skin biopsies showed clear infiltration of host fibroblast cells within the structure of PNPHO-co-TB4 hydrogels; FIG. 8(d). To further confirm fibroblast infiltration and skin extracellular matrix formation within the structure of the inventive composition, skin grafted sites 4 weeks post grafting were stained with Masson's Trichrome. The results in FIG. 9 show collagen fibre formation within the inventive composition 4 weeks post grafting operation. This result confirms the infiltration of fibroblast within the structure of the inventive composition and its potential to integrate with the host tissue and promote neo-dermis formation.

The formation of collagen fibres within the structure of PNPHO-co-TB4 was significantly higher than that of detected within the structure of Integra. The results in CCC showed significantly less collagen formation within the structure of Integra compared with the inventive composition. This result is also in agreement with previous findings reported in the literature (Yiwei Wang, et al., 2015).

The findings from this study confirmed the scaffolding effect of PNPHO-co-TB4 to support host tissue integration, vascular ingrowth, cellular infiltration and neo-dermis formation.

### Technical Feasibility: Conclusion

PNPHO-co-TB4 has been successfully trialled in a powered animal study. The powered animal study involved 40 full-thickness skin grafts with a direct comparison between PNPHO-co-TB4 and the gold standard dermal template (Integra Dermal Matrix). During the study period, all skin grafts treated with the inventive composition survived (100% survival) compared to 82% success rate in Integra group. In addition, the inventive composition expedited vascular network formation, minimised inflammatory response, promoted the infiltration of skin cells within its structure and formed neo-dermis and collagen fibres at the site. These results show the high potential of the technology in scar management post burn injuries.

### Clinical Study—Investigational Device Description

The inventive composition PNPHO-co-TB4 was supplied in a ready-to-use sterile syringe. The product is single use and double pouched. The inventive composition is liquid at room temperature, enabling direct injection to the desired clinical location. At body temperature, the inventive composition forms a white elastic scaffold.

### Device Composition

The inventive composition is an injectable scaffold. There are two main parts in the formulation of the inventive composition; (1) synthetic polymer (PNPHO) and (2) a synthetic non-human or animal-derived peptide, namely Thymosin beta-4. These two parts are chemically bonded resulting in the formation of a single uniform molecule (e.g., PNPHO-co-TB4).

The smart polymer is poly(N-isopropylacrylamide-co-(N-acryloxysuccinimide)-co-(polylactide/2-hydroxy methacrylate)-co-(oligo (ethylene glycol), denoted as PNPHO. The specific formulation of PNPHO that is used in the inventive composition is PNPHO 5:8(5):7:81. Equimolar amount of PNPHO and Thymosin beta-4 are used in the formulation of PNPHO-co-TB4.

### Device Intended Use

PNPHO-co-TB4 is intended to promote bone regeneration. Specifically, PNPHO-co-TB4 is indicated to reduce bone resorption post tooth extraction to enhance patients' health outcome.

### Clinical Investigational Plan—Study Objectives

The primary objective was to identify qualitative measures and analytical methodologies to further investigate the use of the inventive composition. Specifically, assessment and qualification of the healing process and, histological and CT-evaluated bone regeneration in the presence of the inventive composition following tooth extraction compared to an historical (literature) control population. The secondary objective is to examine the in vivo characteristics of the inventive composition in humans.

### Clinical Investigation Design—Clinical Trial End Point

The clinical trial end point of this investigation was to identify qualitative measures and analytical methodologies to further investigate the use of the inventive composition up to 3-4 months post operation. Specifically, assessment and qualification of the healing process and, histological and CT-evaluated bone regeneration in the presence of the inventive composition following tooth extraction compared to an historical (literature) control population. In addition, the clinical trial end point was designed to examine the in vivo characteristics of the inventive composition in humans 3-4 months post-operation.

### Data Quality Assurance

This clinical investigation was designed, constructed and monitored, and data generated, documented, recorded and reported in compliance with ISO 14155, in accordance with SSR's internal procedural requirements, and under Applicant's certified ISO 13485 Quality Management System.

Management of the clinical investigation was outsourced to an independent CRO, Southern Start Research (SSR). All data points were 100% monitored by SSR throughout the trial. Monitoring visits for data Quality Assurance and Quality Control were performed at the intervals specified in Table 7.

TABLE 7
Monitoring visitation schedule

| Clinical Trial | |
|---|---|
| Monitoring Visit | Time point |
| Monitoring visit#1 | Within 10 calendar days of the first participant |
| | treatment (Post visit 3 of participant #1); |
| Monitoring visit#2 | Within 10 calendar days of the fourth participant |
| | treatment (Post visit 3 of participant #4); |
| Monitoring visit#3 | Close-Out Visit after all participants completed the |
| or Close-Out Visit: | study; after Last Participant's Last Visit |

### Participant Population

Ten (10) patients were enrolled in this study. A summary of patient demographics is presented in Table 8.

TABLE 8
Summary of participants' demographic
in PET clinical investigation

| | |
|---|---|
| Number of Participants | N = 10 |
| Number of Male Participants | M = 10 |
| Number of Female Participants | F = 0 |
| Number of participants with smoking history | S = 2 |
| Minimum age at the time of operation | Min-age = 28 |
| Maximum age at the time of operation | Max-age = 73 |
| Median age of participants | Med-age = 51 |
| Average age of participants | Ave-age = 53 |

### Treatment Schedule

Study procedure and schedule for visits and follow ups are outlined in Table 9. The protocol was executed as planned in the clinical investigation plan (CIP). All participants followed the plan and follow-up visits took place as scheduled except for participant #7. Due to personal circumstances, the participant changed his treatment planned and therefore, Histology not collected at Visit 4 for this participant.

### Follow-Up Period

Study participants was followed-up for three (3) months post tooth extraction and treatment with the inventive composition.

TABLE 9
Study procedure and data collection visits

| | Consent & | | Visit 1 (pre- | Visit 2 | Visit 3 | Visit 4 |
|---|---|---|---|---|---|---|
| Study procedure | screening | Enrolment* | op baseline)* | (Tx) | (7 d) | (3 m) |
| Informed consent | ✓ | | | | | |
| Baseline blood sample (for | ✓ | | | | | |
| exclusion criteria) | | | | | | |
| Serum Pregnancy Test | ✓ | | | | | |
| Inclusion/exclusion criteria | | ✓ | | | | |
| Demographics | | ✓ | | | | |
| Medical history | | ✓ | | | | |
| Oral examination | | | ✓ | | ✓ | ✓ |
| Histological examination | | | | | | ✓# |
| CT imaging-referral | ✓@ | | | | ✓@@ | |
| CT imaging-reporting | | | ✓ | | | ✓ |
| Application of PNPHO-co- | | | | ✓ | | |
| TB4 following tooth | | | | | | |
| extraction | | | | | | |
| Photography/Video of | | | ✓ | ✓ | ✓ | ✓ |
| tooth extraction site | | | | | | |
| Concomitant Medication | ✓ | ✓ | ✓ | ✓ | ✓ | ✓ |
| AE/SAE assessment | | | ✓ | ✓ | ✓ | ✓ |

| | |
|---|---|
| Study deviations | As they occur |

*Enrolment and Visit 1 (pre-op baseline) may occur during the same visit (following completion of informed consent process).
#Histological examination of tissue removed from the implant site as part of standard-of-care in preparation for the tooth implant procedure which coincides with Visit 4.
@Referral for the initial CT scan will be made at this visit with the procedure to be performed prior to Visit 1. CT results will be recorded in the CRF at Visit 1.
@@Referral for the second CT scan will be made at this visit with the procedure to be performed within the two week period leading up to Visit 4. CT results will be recorded in the CRF at Visit 4.

Table 10 outlines the intended follow-up visit schedule along with the respective visit windows. Time point zero (0) is the time of tooth extraction (and implantation of the inventive composition for the treatment population).

TABLE 10
Study follow-up visit windows

| | | | |
|---|---|---|---|
| Visit | Window start | Target | Window end |

| | | | |
|---|---|---|---|
| Visit 1 and 2 | — | — | — |

| | | | | | | |
|---|---|---|---|---|---|---|
| Visit 3 | 6 | days | 7 | days | 10 | days |
| Visit 4 | 2 | months | 3 | months | 4 | months |

### Results—Disposal of Subjects and Investigational Device

Ten patients were treated with the inventive composition. The device was implanted in all participants. No investigational device was left at the site post completion of the clinical investigation. All investigational devices were reconciled in accordance with device accountability procedures by Southern Start Research (SSR) clinical research organisation.

All findings are qualitative assessments; these includes but not limited to wound appearance at different time points post-extraction, reports of pain and discomfort by patients, radiological appearance of underlying bone with CT-scans three months after operation, visual appearance of the site during implant placement operation and histological examination of the treatment site three-four months after operation.

### Device Usability

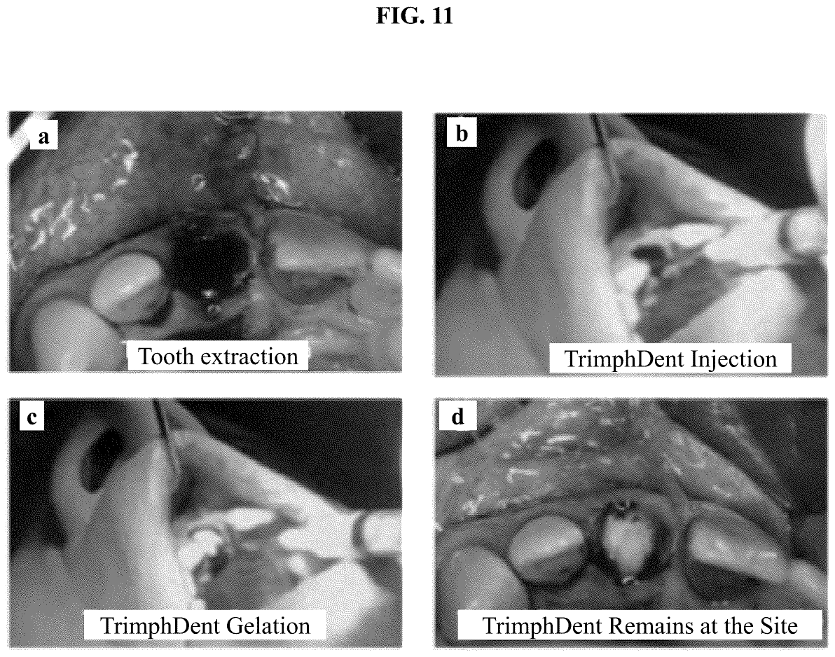

There was no report of device malfunction throughout the clinical investigation. The inventive composition was provided in a ready-to-use format; this negates the need for pre-mixing and any other preparation step by the clinicians prior to the operation. FIG. 11(b), in particular, shows that the inventive composition can be readily injected into the extraction site through a 21 G needle. Subsequently, due to the hydrophilic nature of the product, the inventive composition mixes with blood at the site and forms a hydrogel to stabilise the clot.

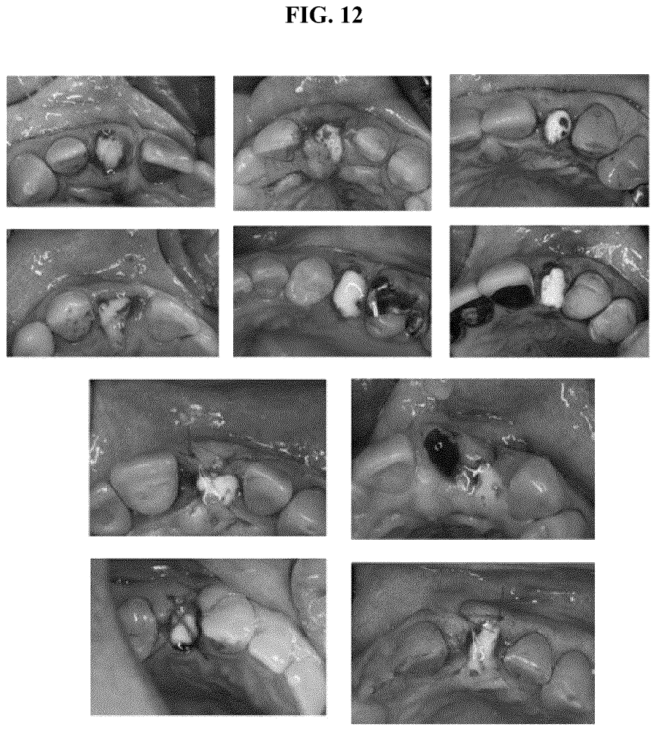

The clinical use of the device in PET trial showed that the inventive composition injection into the socket site was successful for all 10 patients and the Principal Investigator did not report any device-malfunction. Results in FIG. 12 showed 100% successful rate in the application and gelation of the inventive composition for all 10 patients.

TABLE 11
Usability of the inventive composition in PET trial for 10 patients

| Patient | Injection/ | | | Mem- | |
|---|---|---|---|---|---|
| # | Application | Gelation | Adhesion | branes | Containment |
| 001-001 | Successful | Instantly | Successful | Not used | No |
| | | | | | Containment |
| 001-002 | Successful | Instantly | Successful | Not used | No |
| | | | | | Containment |
| 001-003 | Successful | Instantly | Successful | Not used | No |
| | | | | | Containment |
| 001-004 | Successful | Instantly | Successful | Not used | No |
| | | | | | Containment |
| 001-005 | Successful | Instantly | Successful | Not used | No |
| | | | | | Containment |
| 001-006 | Successful | Instantly | Successful | Not used | No |
| | | | | | Containment |
| 001-007 | Successful | Instantly | Successful | Not used | No |
| | | | | | Containment |
| 001-008 | Successful | Instantly | Successful | Not used | No |
| | | | | | Containment |
| 001-009 | Successful | Instantly | Successful | Not used | No |
| | | | | | Containment |
| 001-010 | Successful | Instantly | Successful | Not used | No |
| | | | | | Containment |

A summary of findings from the usability of the inventive composition is outlined in Table 11. According to the Principal Investigator, the use of the inventive composition can save up to 45 minutes in the theatre. This can be achieved as the use of the inventive composition post extraction negates the need for primary closure, e.g., the use of membranes and micro-suturing of the site.

TABLE 12
Safety of the inventive composition post tooth extraction based
on visual observation and histochemical analyses of the sites

| | In all follow up visits up-to 3 | | 3 months post administration of | | |
|---|---|---|---|---|---|
| | months post administration | | inventive composition | | |
| | of the inventive composition | | CT-scans | | |
| | | Visual | Appearance of | | |
| | Device | observation | the site with CT- | | |
| | related | (infection, | scans (infection, | Histochemical analyses | |
| | adverse | inflammation | inflammation | | Foreign body |
| Patient # | event | or normal) | or normal¹) | Synopsis | reaction |
| 001-001 | No | Normal | Normal | No | No |
| 001-002 | No | Normal | Normal | No | No |
| 001-003 | No | Normal | Normal | No | No |
| 001-004 | No | Normal | Normal | No | No |
| 001-005 | No | Normal | Normal | No | No |
| 001-006 | No | Normal | Normal | No | No |
| 001-007 | No | Normal | Not Applicable | Not Applicable | Not Applicable |
| 001-008 | No | Normal | Normal | No | No |
| 001-009 | No | Normal | Normal | No | No |
| 001-010 | No | Normal | Normal | No | No |

¹Identification as Normal in the CT-scan results denotes the absence of inflammation, infection, necrosis, hypertrophic bone growth, hypertrophic fibrosis

### Device Safety and Wound Healing

All ten patients treated with the inventive composition returned for the first follow-up visit, one-week post operation. There was no report of pain or discomfort from any of the patients. During the oral examination (one-week post administration), there was no sign of infection or inflammation at the site. In addition, wound closure and soft tissue formation were examined by the Principal Investigator. In all ten patients, wound closure was noted and expedited soft tissue formation was detected (results in FIG. 12).

Three months post tooth extraction and treatment with the inventive composition, patients underwent implant placement procedure. At this point, biopsies were collected from the inventive composition's injection site. Samples were histochemical analysed by an independent laboratory (Sonic Clinical Trials Pty Ltd). Findings from these histochemical analyses are summarised in Table 12. In all samples analysed, there was no evidence of necrosis, foreign body type giant cell or foreign body reactions. These findings suggest that the inventive composition is well-tolerated in the body and biocompatible in vivo.

### Indications of Efficacy on Bone Regeneration

Three months post operation, CT-scan imaging of the site was used to investigate the healing progress and the extend of bone resorption at the site. Independent CT-scan reports from Dr Tom Huang at Envision Medical Imaging confirmed that the bone resorption was minimised. In addition to CT-scan imaging of the site, during the implant placement procedure, biopsies were collected from the inventive composition injection sites. The samples were fixed and sent for independent histochemical analysis. Hematoxylin and Eosin (H&E) and Masson's Trichrome straining of the sites were used to analyses the pathological behaviour at the site and the bone regeneration process. In all analysed samples, histochemical analyses showed the formation of interconnected viable bony trabeculae, a mixture of woven and lamellar bone as well as active osteoblasts and osteoclasts. In all patients, active periodontal bone remodelling was noted.

All reports from independent laboratories on H&E and Mason's trichrome stained histochemical specimen is presented in Table 13.

TABLE 13
Summary of findings from H&E and Masson's trichrome
staining of sites tested with the inventive composition

| | Macroscopic | | |
|---|---|---|---|
| Patient # | Findings | Histology Microscopic findings | Diagnosis of the site |
| 001-001 | Biopsy fresh | Sections show the specimen is composed of | PERIODONTAL- |
| | tissue. The | interconnected viable bony trabeculae, which are a | BONE-Reactive |
| | specimen consists | mixture of woven and lamellar bone, confirmed on | host bone |
| | of a core of bone | polarisation and Masson staining. The trabeculae are | remodelling |
| | measuring 4 mm | rimmed by a mixture of osteoclasts and osteoblasts, | |
| | in length by 2 mm | consistent with ongoing remodelling. There is no | |
| | in diameter. | evidence of necrosis of a foreign body reaction and | |
| | | no specific pathological abnormality is identified. | |
| | | The appearance is keeping with reactive host bone | |
| | | remodelling. | |
| 001-002 | Biopsy fresh | Sections show the specimen is composed of | PERIODONTAL- |
| | tissue. The | interconnected viable bony trabeculae, which are a | BONE-Reactive |
| | specimen consists | mixture of woven and lamellar bone, confirmed on | host bone |
| | of a core of bone | polarisation and Masson staining. The trabeculae are | remodelling |
| | measuring 8 mm | rimmed by a mixture of osteoclasts and osteoblasts, | |
| | in length by 2 mm | consistent with ongoing remodelling. There is no | |
| | in diameter. | evidence of necrosis or a foreign body reaction and | |
| | | no specific pathological abnormality is identified. | |
| | | The appearances are keeping with reactive host bone | |
| | | remodelling. | |
| 001-003 | Biopsy fresh | Sections show the specimen is composed of | PERIODONTAL- |
| | tissue. The | interconnected viable bony trabeculae, which are a | BONE-Reactive |
| | specimen consists | mixture of woven and lamellar bone, confirmed on | host bone |
| | of a core of bone | polarisation and Masson staining. The trabecular are | remodelling |
| | measuring 12 mm | rimmed by a mixture of osteoclasts and osteoblasts, | |
| | in length by 3 mm | consistent with ongoing remodelling. There is no | |
| | in diameter. | evidence of necrosis or a foreign body reaction and | |
| | | no specific pathological abnormality is identified. | |
| | | The appearance is in keeping with reactive host bone | |
| | | remodelling. | |
| 001-004 | The specimen | Sections show a fragment of tissue comprising | PERIODONTAL- |
| | consists of a core | variably sized trabeculae of viable bone which has a | BONE-Viable |
| | of bone | sclerotic appearance and is composed of a mixture of | sclerotic bone with |
| | measuring 4 mm | woven and lamellar bone. Focal ongoing remodelling | ongoing remodelling, |
| | in length by 2 mm | with an osteoblastic lining is seen and occasional | no foreign material |
| | in diameter. | resorption pits are included. Accompanying bone | seen |
| | | dust is seen. Small fragments of crushed | |
| | | fibrovascular tissue is included in which a | |
| | | lymphocytic infiltrate is noted however foreign | |
| | | material is not identified and a foreign body type | |
| | | giant cell reaction is not noted. Features to suggest | |
| | | sepsis are not seen. There is no evidence of | |
| | | malignancy. | |
| 001-005 | Specimen site not- | Sections confirms the presence of a core of bone | PERIODONTAL- |
| | indicated on | much of which is fragmented and which is composed | BONE-Viable |
| | container. The | of sclerotic bone comprising woven and lamellar | sclerotic bone with |
| | specimen consists | bone, all of which is viable in nature and in which a | remodelling, no |
| | of a core bone | degree of ongoing remodelling with osteoblastic and | foreign material |
| | measuring 6 mm | focally osteoclastic activity, manifest as resorption is | noted |
| | in length by 2 mm | noted. Accompanying slightly crushed fibrovascular | |
| | in diameter. | stroma is included which a patchy lymphocytic | |
| | Embedded whole. | infiltrate is noted and a rare hemosiderin laden | |
| | | macrophage is present. Foreign material is not seen | |
| | | and a foreign body giant cell reaction is not | |
| | | identified. Features to suggest sepsis are no evident. | |
| | | There is no evidence of malignancy. | |
| 001-006 | Specimen site not- | Sections show a core of bone composed of | PERIODONTAL- |
| | indicated on | interconnected trabeculae of viable sclerotic mature | BONE-Sclerotic |
| | container. The | lamellar and woven bone is included with intervening | viable bone with |
| | specimen consists | loose fibrovascular tissue and bone dust. Evidence of | evidence of pre- |
| | of a core of bone | prior remodelling with prominent cement lines with | remodelling, no other |
| | measuring 5 mm | scattered bland osteoblasts are present and the | abnormality noted |
| | in length by 2 mm | osseous trabeculae are viable throughout. Bone dust | |
| | in diameter. | is included however convincing foreign material and | |
| | Embedded whole | foreign body type giant cell reaction is not noted. | |
| | (1 block) RA12 | Features to suggest sepsis are not seen. There is no | |
| | | evidence of malignancy. | |
| 001-007 | Not applicable | Not applicable | Not applicable |
| 001-008 | Specimen site not- | Sections confirm the presence of a core of bone | PERIODONTAL- |
| | indicated on | composed of mature lamellar and somewhat compact | BONE-Viable |
| | container. The | bone merging with slightly thickened trabecular in | bone with ongoing |
| | specimen consists | which a combination of both woven and lamellar | remodelling |
| | of a core of bone | bone is seen in keeping with a degree of remodelling. | No other |
| | measuring 5 mm | The osseous component is viable throughout with | abnormality seen |
| | in length by 2 mm | osteoblastic and osteoclastic activity is noted. A | |
| | in diameter. | foreign material is not seen and featured to suggest | |
| | Embedded whole. | sepsis are not identified. There is no evidence of | |
| | (1 block) RF2 | malignancy | |
| 001-009 | Biopsy fresh | Sections confirm the presence of a core of bone much | PERIODONTAL- |
| | tissue. The | of which comprises compact mature lamellar bone of | BONE- |
| | specimen consists | cortical origin merging with interconnected | Cortical and |
| | of a core of bone | trabeculae of bone with a more cancellous | cancellous bone with |
| | measuring 6 mm | appearance which is moderately thick centrally | evidence of ongoing |
| | in length by 2 mm | composed of woven bone but exhibiting peripheral | remodelling foreign |
| | in diameter. | lamination in keeping with maturation. Focally a | body reaction not |
| | | degree of ongoing remodelling with osteoblastic | noted no sepsis |
| | | activity is noted. The companying stroma has a loose | |
| | | fibrovascular appearance. The bone throughout is | |
| | | viable with osteocytes visible within the lacunae. A | |
| | | foreign body type giant cell reaction is not seen. | |
| | | There is no evidence of sepsis. There is no evidence | |
| | | of malignancy. | |
| 001-010 | | Section confirm the presence of a core bone in which | PERIODONTAL- |
| | | an elongated strip of compact lamellar bone is | BONE-viable |
| | | included, bordered at its perimeter by reactive | woven bone and |
| | | appearing woven bone which itself can be seen to be | mature lamellar bone |
| | | undergoing lamellation at the perimeter and which | no evidence of |
| | | mergers with bone with more cancellous architecture | foreign material no |
| | | but composed of interconnected trabecular of largely | foreign body reaction |
| | | woven bone within a loose, slightly oedematous and | no evidence of sepsis |
| | | well vascularised stroma with dilated thin walled | |
| | | vascular channel. A degree of bone remodelling | |
| | | within area of osteoclastic and osteoblastic activity | |
| | | are seen. The osseous components is viable | |
| | | throughout, foreign material is not and foreign body | |
| | | type giant cell reaction is not noted. Features suggest | |
| | | sepsis are not identified. The Masson trichrome stain | |
| | | confirms these findings. There is no evidence of | |
| | | malignancy. | |

### Aerosol Formation Capabilities of PNPHO-Co-TB4

Particle size analysis as volume-diameter distribution and derived parameters (d10, d50, d90 and % V>10) for each formulation with different solid content (PNPHO concentration between the range of 17.5 mg/mL to 140 mg/mL) was carried out. The aerosol droplets generated from all formulations presented a bimodal distribution, regardless the concentration of the formulation and temperature tested. Generally, the first peak (˜50 μm) tends to increase with the decreasing concentration of the formulation whereas the second peak (˜500-600 μm) increases with the concentration. This suggests that the majority of the aerosol droplets were between the 10-100 μm range.

The percentile sizes for all the diluted formulations were 21.4±3.9 μm, 45.6±9.4 μm and 238.6±181.5 μm (on average) for d10, d50 and d90, respectively. The prevalent median size for nasal delivery is between 30 and 120 μm (1) and therefore all diluted formulations were within the specification for nasal delivery. Importantly, all formulations had less than 3% volume of droplets with diameter <10 μm, suggesting their suitability for nasal delivery and avoiding lower airways deposition. The 90th percentile size presented the greatest variability, suggesting potential issues with coarse droplets emission, especially for the more concentrated formulations.

### Plume Coverage

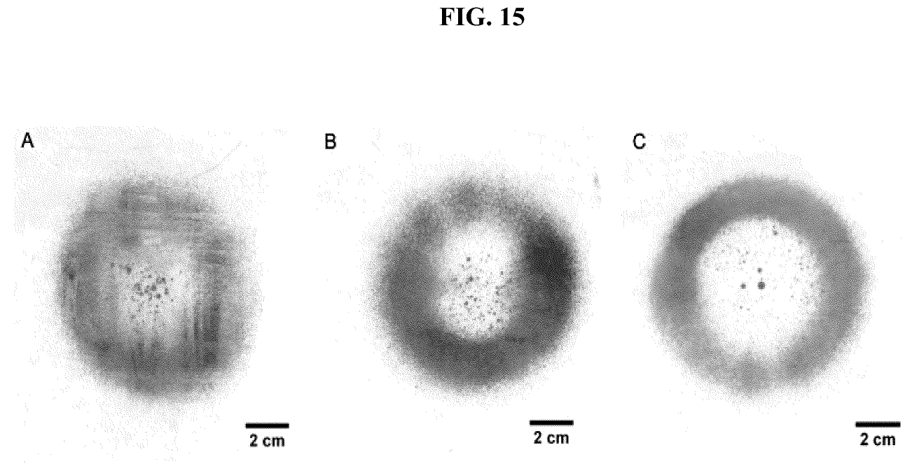

The characterisation of the spray pattern of nasal formulations is recommended by the FDA. The plume pattern for the tested formulations (actuated at room temperature) is represented in FIG. 15. All formulations showed a wide coverage area, but the most evident feature was the lack of deposition at the center of the plume, with the increase of the formulation concentration.

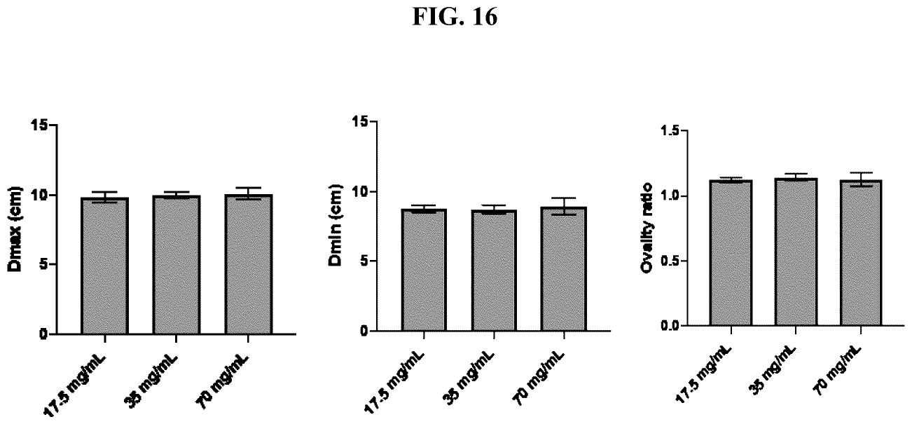

Despite these differences, the Dₘₐₓ and Dₘᵢₙ values (FIG. 16) were constant across samples and the obtained ovality ratios (˜1) are indicative of spray symmetry upon aerosolisation. Ovality ratios for all formulations are within the FDA specifications (1.00-1.30). In practice, the best option for nasal delivery should be a trade-off between coverage area and improved residence time. A higher coverage area could not provide the best outcomes if runoff occurs.

### Nasal Deposition Pattern

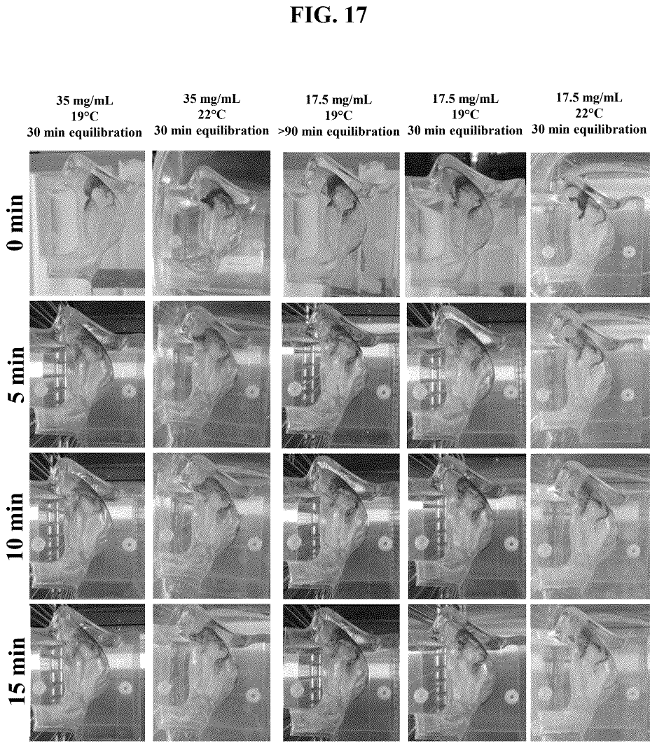

The deposition patterns of the formulations on the human nasal model are depicted in FIG. 17. Both formulations (17.5 mg/mL and 35 mg/mL) tested showed a rapid adhesion, as the deposition patterns remained stable and relatively unchanged from the actuation and up to 15 minutes. Further, no dripping into the throat was observed. The adhesiveness of the formulation at short time periods may indicate higher residence times and bioavailability of the delivered cargos. Both formulations also were able to reach the olfactory region (the upper part of the nasal region), which is important for nose-to-brain delivery.

In Vitro Release of Drugs from PNPHO-Based Hydrogel

Ciprofloxacin HCl is used as a model drug to investigate the potential of PNPHO based hydrogels to control the release of small hydrophilic drugs and to prevent drug burst release post administration. The assembly of the hydrophobic domains in the polymer chain during the gelation process causes water to be expelled from the matrix. To assess the amount of ciprofloxacin HCl being leached out from the PNPHO-co-TB4, hydrogels were assessed using a snapwell setup. Briefly the ciprofloxacin HCl powder was dissolved (20 mg/mL) in the PNPHO-co-TB4 solution. Subsequently 200 μL of the PNPHO-co-TB4/ciprofloxacin HCl formulation was placed into the apical chamber of the snapwells and allowed to form a hydrogel whilst the snapwells were standing on a flat surface to avoid polymer loss through the membrane, at 37° C. for 30 min. Next the top liquid supernatant layer of the hydrogels was collected.

To further analyse the drug release from the gel over time, 2 mL of PBS was added to the basolateral compartment of each snapwell before the plates were incubated at 37° C. under constant orbital shaking (60 rpm). Samples were withdrawn (200 μL) from the basolateral media at predetermined time points for 24 h, and replaced with an equal amount of fresh pre-warmed PBS each time. All samples were quantified for ciprofloxacin HCl using and a validated high-performance liquid chromatography method (HPLC, Shimadzu, Sydney NSW, Australia).

### Quantification of Ciprofloxacin HCl Via HPLC

Quantification of ciprofloxacin HCl was determined using an HPLC system consisting of an LC20AT pump, SIL20AHT autosampler and SPD-20A UV-VIS detector (Shimadzu, Sydney NSW, Australia). Sample quantification was performed using a reverse-phase Luna C-18 Column (Phenomenex, Torrance, USA) 150×4.6 mm and 3 μm particle size. Measurements were carried out using a mobile phase consisting of phosphate buffer (pH 7.2):acetonitrile (75:25 v/v), a flow rate of 0.7 mL/min, a detection wavelength of 275 nm and injection volume of 100 μL. Standard solutions were prepared fresh daily in needle wash of acetonitrile:water (50:50 v/v) and linearity confirmed within the concentration of 0.05-100 μg/mL with a regression value >0.999.

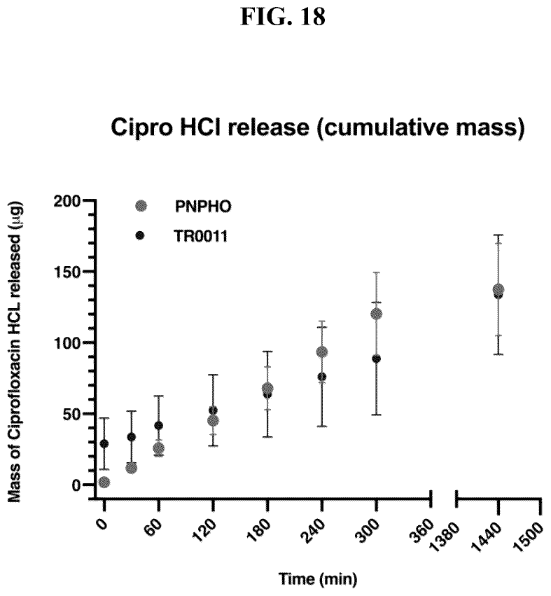

The initial release of ciprofloxacin HCl from the PNPHO hydrogels during the gelation process (30 min) under physiological conditions was evaluated. It was found that an average of 2.7±1.4 μg and 28.9±17.9 μg (n=3) (FIG. 18; t=0) of cipofloxacin HCl was released into the liquid apical layer of PNPHO and PNPHO-co-TB4, respectively. It is important to note that the initial release of the tested drug from the hydrogels is negligible in comparison with the loaded amount, i.e., 20 mg/mL loaded and 2.7±1.4 μg and 28.9±17.9 μg released from PNPHO and PNPHO-co-TB4, respectively.

The relatively low percentage of ciprofloxacin release upon administration from the hydrogels display the high potential of the invention for drug delivery applications. The release of ciprofloxacin HCl from the hydrogels over time at 37° C. is depicted as cumulative mass in each formulation in FIG. 18. There was no significant difference between the amounts of ciprofloxacin HCl released between the two hydrogels, with 137.4±32.4 μg (89.5±15.8%) and 133.7±42 μg (65.3±10.5%) released from PNPHO and PNPHO-co-TB4 after 24 h, respectively. These results confirmed sustained and controlled release profile of the drug from PNPHO based hydrogels.

### Summary of Adverse Events

There has been no medical device adverse event reported throughout the clinical trial. The only adverse event reported in this trial was related to the treatment plan of participant number 7; histology was not collected at Visit 4 for this participant as the participant elected to change his treatment plan precluding bone histology.

### Conclusion on Device Usability

The inventive composition was administered to all ten patients with no difficulty. No device (syringe) malfunction was reported in the study. There was no need for preparation and/or mixing of the device prior to use. The inventive composition instantly forms a white hydrogel upon injection to the socket site. The product mixes with the blood and adhered to the extraction site. The product adhered to the site and there was no need for primary closure at the site to contain the device.

### Device Safety

No device related adverse event, or serious adverse event was reported in the study. All ten (10) patients returned for the first follow-up visit, 7 days post tooth extraction, there was no report of inflammation, infection, pain or discomfort from the patients Nine (9) patients underwent implant placement operation during which native tissues were collected for histochemical analyses. H&E staining of the site showed that the product is well-tolerated in the body and there was no sign of abnormality or foreign body giant cells at the site. No necrosis or necrotic tissue was noted at the site. In addition, CT-scans of the extraction sites and the inventive composition treatment site showed that there is no osseous abnormality at the site.

### Device Efficacy

Wound closure was noted for all patients 7 days post-tooth extraction and treatment with the inventive composition. Cellular-level, osteoblastic and osteoclastic activity, active bone remodelling was noted. Viable bone and bone remodelling were noted at the treatment site, showing osteoconductive properties of the device. The product did not induce bone formation and did not have any other phenotypic effect on the host tissue as hypothesised.

### Assessment of Risk and Benefit

The potential benefits of the device and the residual risks identified have been evaluated on an individual and collective basis to determine whether or not they are acceptable when weighed against the potential benefits of the device.

Emerging evidence suggests bone anchored prosthetics improve quality of life measures including mastication, speech and general health outcomes. However, economic barriers and access to oral surgeons are recognised as limitations to widespread use of implant secured prosthetics. From a clinical perspective, minimising the loss of bone after tooth extraction is imperative to simplify and thus improve access to bone anchored prosthetics.

Loss of bone volume after tooth extraction is hard to manage for clinicians and imposes a financial burden on patients and the health system, particularly in rural and socioeconomically disadvantaged areas. The extent of this problem and challenge in dentistry is highlighted by the fact that one in every two patients requires secondary grafting procedure to increase bone volume for successful implant placement. The inventive composition is an easy to use material that can preserve ridge bone volume after tooth extraction. Dentists appreciate the ease of use and the predictability of implant placement procedure. Patients will benefit as the use of the inventive composition promotes wound healing after tooth extraction, accelerates bone healing, and may potentially prevent the need for a secondary augmentation procedure.

Application of the inventive composition in fresh extraction sockets was simple and easy to use in clinical practice. Unlike all other bone substitutes, the inventive composition was delivered to a socket as a liquid and forms an elastic matrix at the site. The primary aim pilot PET trial was to investigate safety, usability and osteoconductivity of the inventive composition. Markers of efficacy including radiological imaging and histochemical analysis were collected. In summary, while the trial was not powered, PET confirmed the safety and usability of the device; no device malfunction was noted and no device-related adverse event was detected.

Given consideration to the intended use, intended user, reasonably foreseeable misuse, the impact use of the he inventive composition device family may have, and with consideration of the generally acknowledged state of the art, it can be concluded that the risk management process has confirmed the potential device benefits outweigh the residual risks.

### Clinical Relevance of the Inventive Composition

The inventive composition has been developed as a safe and easy-to-use biomaterial to fill a tissue defect/cavity. The inventive composition is a single uniform molecule comprised of a synthetic “smart” polymer (PNPHO) that is crosslinked with Thymosin beta-4. The inventive composition is liquid at room temperature, enabling direct injection to the desired clinical location. The inventive composition forms an elastic gel on exposure to body temperature, mixes with blood and stabilises the clot at the site. Ten (10) patients were administered with the inventive composition. No device malfunction or device related adverse event were reported or noted upon the use of the inventive composition. Wound healing was noted one week after administration.

Three months post-operation, no pathological abnormality, e.g., inflammation, infection or giant cells were detected at the administration site. In addition, active bone remodelling as well as osteoblast and osteoclast activity was detected at the sites treated with the inventive composition, confirming osteoconductive properties of the device. These findings showed the high potentials of the inventive composition for wound healing in both soft and hard tissue as the product did not induce any phenotypic effect, e.g., soft and hard tissue repair at the site. However, further health benefits are thought to be achievable by increasing the osteoinductive properties of the inventive composition.

### SUMMARY OF CLINICAL STUDY

The pilot trial involved the use of the inventive PNPHO-co-TB4 scaffold for socket preservation post tooth extraction in ten patients. In all patients, Applicant's device was successfully administered; there was no need for use of membrane or micro-suturing at the extraction site. This allows the Principal Investigator to save time in the theatre. In follow-up visits, wound closure was noted at seven days post-extraction and there was no sign of infection or inflammation in any patients. Three months post use of the inventive composition, tissues biopsies were collected from the site for histochemical analyses; the results showed that the product was fully resorbed and there was no sign pathological abnormality at the site. In addition, active bone remodelling was also noted at the site.

Although the invention has been described with reference to specific examples it will be appreciated by those skilled in the art that the invention may be embodied in many other forms.

Applicant acknowledges the contribution of Dr Hui Ong and Dr Dina Silva in performing in vitro aerosol formation studies and benchtop drug release tests.

1. A composition comprising a polymer and a natural or synthetic peptide or protein (NSPP), wherein the polymer comprises:
a first monomer for binding water,
a second monomer for imparting mechanical properties to said hydrogel;
a third monomer for binding to a natural or synthetic peptide or protein (NSPP); and
a fourth monomer for imparting phase-transition behaviour,
and wherein the natural or synthetic peptide or protein (NSPP) is Thymosin beta-4 or a functional homolog thereof.
